(19) United States
(12) Patent Application Publication
(10) Pub. No.: US 2019/0249261 A1
(43) Pub. Date: Aug. 15, 2019

(54) Methods For Identifying Clonal Mutations And Treating Cancer

(71) Applicant: MAYO FOUNDATION FOR MEDICAL EDUCATION AND RESEARCH, Rochester, MN (US)

(72) Inventors: David DINGLI, Rochester, MN (US); Benjamin WERNER, London (GB); Ame TRAULSEN, Plon (DE)

(73) Assignee: MAYO FOUNDATION FOR MEDICAL EDUCATION AND RESEARCH, Rochester, MN (US)

(21) Appl. No.: 16/300,987

(22) PCT Filed: May 12, 2017
(86) PCT No.: PCT/US17/32537

§ 371 Date: Nov. 13, 2018

Related U.S. Application Data
(60) Provisional application No. 62/335,298, filed on May 12, 2016.

(51) Int. Cl. C12Q 1/6886; C12Q 1/6827; G16H 50/50; G16H 50/20; G16H 50/70

(52) U.S. Cl. CPC C12Q 1/6886; C12Q 1/6827; G16H 50/50; C12Q 2600/156; G16H 50/70; C12Q 2600/106; G16H 50/20

(57) ABSTRACT

The invention provides methods for establishing which genomic alterations are truly clonal in cancer, and for determining the confidence for calling true clonal mutations in individual tumors using multi-region sequencing data. The invention further provides methods for treating cancer that comprise confidently determining truly clonal genomic alternations in the target cancer that is to be treated.

### FIELD OF THE INVENTION

The invention provides methods for establishing which genomic alterations are truly clonal in cancer, and for determining the confidence for calling true clonal mutations in individual tumors using multi-region sequencing data. The invention further provides methods for treating cancer that comprise confidently determining truly clonal genomic alternations in the target cancer that is to be treated.

### BACKGROUND OF THE INVENTION

Recent advances in next-generation sequencing have led to the widespread identification of somatic changes in the genomes of a large number of tumors, raising the hope to transform cancer therapy (1). Novel treatments aim at targeting specifically cancer genomic alterations that drive the growth of individual tumors, promising a future of personalised cancer medicine (2-7; Nicholas McGranahan et al. Clonal neoantigens elicit T cell immunoreactivity and sensitivity to immune checkpoint blockade. Science 351:1463-1469 (2016); Palucka, A. K. & Coussens, L. M. The Basis of Oncoimmunology. Cell 164, 1233-1247 (2016); van der Burg, S. H., Arens, R., Ossendorp, F., van Hall, T. & Melief, C. J. M. Vaccines for established cancer: overcoming the challenges posed by immune evasion. Nature Reviews Cancer 16, 219-233 (2016); Schumacher, T. N. & Schreiber, R. D. Neoantigens in cancer immunotherapy. Science 348, 69-74 (2015)).

Success however, relies on selecting the correct tumor-driving alterations to target in each patient (8; Yates L R, Campbell P J (2012) Evolution of the cancer genome. Nature Reviews Genetics 13(11):795-806; Sawyers, C. L. The cancer biomarker problem. Nature 452, 548-552 (2008); Bedard, P. L., Hansen, A. R., Ratain, M. J. & Siu, L. L. Tumour heterogeneity in the clinic. Nature 501, 355-364 (2013)), a task that is complicated by the following challenges: (i) the number of genetic alterations in a given tumor can be very large, and most of them are likely “passenger” mutations that do not drive malignant growth (1), (ii) there is considerable variation in the mutational landscape across patients (1) and (iii) within-tumor genetic diversity is very high (9, 10). In particular, intra-tumor heterogeneity (ITH) represents a crucial problem for cancer therapy because effective targeted approaches rely on selectively striking mutant genes that are present in all cells of the tumor (11-13). Therefore, establishing which genomic alterations are truly clonal in the tumor is of critical importance.

Because of ITH, single tumor biopsies introduce an important bias in detecting clonal alterations, hence the need for multi-region profiling of distinct tumor samples (14-16). Multi-region sequencing allows reconstructing phylogenetic trees describing the tumor evolutionary history (17-20). If a mutation is clonal, it must appear in the “trunk” of the tree. However, the opposite is not necessarily true. This is because, despite the enormous improvement of multi-region profiling, often the proportion of cancer cells that is ultimately sampled from the potentially hundreds of billions of cells that form the tumor, is very small. A mutation that appears truncal in the “sampled” tree, may still be subclonal in the whole tumor, simply because we cannot profile every cell in the tumor (18, Williams M J, Werner B, Barnes C P, Graham T A, Sottoriva A (2016) Identification of neutral tumor evolution across cancer types. Nature Genetics 48:238-244; Sottoriva A, et al. (2015) A Big Bang model of human colorectal tumor growth. Nature Genetics. doi:10.1038/ng.3214.), see also FIG. 5. Taking more, larger or spatially distant samples can mitigate the problem, but the fundamental question remains: how confident are we that a truncal mutation in a sample is a true clonal mutation in the tumor?

Thus, there remains a need for establishing which genomic alterations are truly clonal in the tumor, and for determining the confidence for calling true clonal mutations in individual tumors using multi-region sequencing data.

### SUMMARY OF THE INVENTION

The invention provides methods for establishing which genomic alterations are truly clonal in cancer, and for determining the confidence for calling true clonal mutations in individual tumors using multi-region sequencing data. The invention further provides methods for treating cancer that comprise confidently determining truly clonal genomic altemations in the target cancer that is to be treated.

Thus, in one embodiment, the invention provides a method for identifying a true clonal mutation in cancer cells from a mammal for use in treating said mammal, comprising, A) generating whole exome sequencing data (wherein whole exome sequencing is sequencing of all expressed genes or a portion of the expressed genes, more preferably a portion of sequenced genes representing a majority of the expressed genes), from at least three samples of cancer cells obtained from different regions in said mammal (wherein said different regions may be different regions in the same tumor, may be different regions of a biopsy, may be from different biopsies, etc.), comparing said sequencing data to a corresponding wild-type reference genes in a databank, and thereafter: i) Identifying the mutated genes in common between all three samples and identifying mutated genes that are in common between any two sample combinations, (which may be any combination of two samples of all three samples, ii) determining whether said mutated genes in common between all three samples are the same as the mutated genes in common between said two sample combinations, wherein when the number of mutated genes in common with all three samples is equal to or greater than the number of mutated genes in common between said two sample combinations then mutated genes in common in all three samples are true clonally mutated genes, then proceed to step B), otherwise proceed to step iii), iii) generate whole exome sequencing data for an additional sample, e.g. a fourth sample, and identifying mutated genes in common between all samples, e.g. four samples, and identifying mutated genes that common between combinations of any of the one less than all four samples, e.g. three samples, and iv) determining whether said mutated genes in common between all, e.g. four, samples are the same as the mutated genes in common between said combinations of one less than all samples, e.g. combinations of three samples from all four samples, wherein when the number of mutated genes in common in all samples is equal to or greater than the number of mutated genes in common between the combinations of one less than all samples, then proceed to step B), otherwise repeat steps iii) and iv), B) administering to said mammal a therapeutically effective amount of a compound targeting said cancer cells containing at least one said clonally mutated gene for treatment of said cancer cells. While the specific numbers of samples (2, 3, 4) have been specified above, it is not intended that the present invention be limited to a specific sample number; indeed, the present invention contemplates sample numbers selected from the group consisting of 5, 6, 7, 8, 9, 10, 11 and 12 (or higher).

In one embodiment, said at least one of the identified true clonally mutated genes is a tyrosine kinase gene. In one embodiment, said compound is a tyrosine kinase inhibitor. In one embodiment, said at least one of the identified true clonally mutated genes is an epidermal growth factor receptor (EGFR) gene and wherein said at least one of the identified true clonally mutated genes is not a K-Ras (KRAS) gene. Thus, in a further embodiment, said compound is an anti-EGFR1 therapeutic monoclonal antibody. In one embodiment, said treatment slows cancer cell growth in said patient. In one embodiment, said treatment kills cancer cells in said patient.

Thus, in one embodiment, the invention provides a method for identifying a true clonal mutation in cancer cells from a mammal for use in treating said mammal, comprising, A) generating whole exome sequencing data, from at least two samples of cancer cells obtained from different regions in said mammal, comparing said sequencing data to corresponding wild-type reference genes in a databank, and thereafter: i) identifying the mutated genes in common between both samples and identifying mutated genes in either of the two samples, ii) determining whether said mutated genes in common between both samples are the same as the mutated genes in either of said two samples, wherein when the number of mutated genes in common in both samples is equal to or greater than the number of mutated genes in either of said two samples then mutated genes in common in both samples are true clonally mutated genes, then proceed to step B), and B) administering to said mammal a therapeutically effective amount of a compound targeting said cancer cells containing at least one said true clonally mutated gene for treatment of said cancer cells.

Thus, in one embodiment, the invention provides a method for identifying a true clonal mutation in cancer cells from a mammal for use in treating said mammal, comprising, A) generating whole exome sequencing data from at least three samples of cancer cells obtained from different regions in said mammal, comparing said sequencing data to corresponding wild-type reference genes in a databank, and thereafter: i) identifying the mutated genes in common between all three samples and identifying mutated genes that are in common between any two sample combinations, and ii) determining whether said mutated genes in common between all three samples are the same as the mutated genes in common between said two sample combinations, wherein when the number of mutated genes in common with all three samples is equal to or greater than the number of mutated genes in common between said two sample combinations then mutated genes in common in all three samples are true clonally mutated genes, then proceed to step B), and B) administering to said mammal a therapeutically effective amount of a compound targeting said cancer cells containing at least one said true clonally mutated genes for treatment of said cancer cells. In one embodiment, said at least one of said identified true clonally mutated genes is a tyrosine kinase gene. In one embodiment, said compound is a tyrosine kinase inhibitor. In one embodiment, said at least one of said true clonally identified genes is an epidermal growth factor receptor (EGFR) gene and wherein at least one of said true clonally identified mutated genes is not a KRAS gene. In one embodiment, said compound is an anti-EGFR1 therapeutic monoclonal antibody. In one embodiment, said treatment slows cancer cell growth in said patient. In one embodiment, said treatment kills cancer cells in said patient. In one embodiment, wherein when the number of mutated genes in common with all three samples is less than the number of mutated genes in common between said two sample combinations then generating whole exome sequencing data from an additional sample of cancer cells obtained from different regions in said mammal, comparing said sequencing data to corresponding wild-type reference genes in a databank, and thereafter: iii) identifying the mutated genes in common between all four samples and identifying mutated genes that are in common between any three sample combinations, iv) determining whether said mutated genes in common between all four samples are the same as the mutated genes in common between said three sample combinations, wherein when the number of mutated genes in common with all four samples is equal to or greater than the number of mutated genes in common between said three sample combinations then mutated genes in common in all four samples are true clonally mutated genes, then proceed to step B).

Thus, in one embodiment, the Invention provides a method for identifying a true clonal mutation in cancer cells from a mammal for use in treating said mammal, comprising, A) generating whole exome sequencing data, from at least four samples of cancer cells obtained from different regions in said mammal, comparing said sequencing data to corresponding wild-type reference genes in a databank, and thereafter: i) Identifying the mutated genes in common between all four samples and identifying mutated genes that are in common between any three sample combinations, ii) determining whether said mutated genes in common between all four samples are the same as the mutated genes in common between said three sample combinations, wherein when the number of mutated genes in common with all four samples is equal to or greater than the number of mutated genes in common between said three sample combinations then mutated genes in common in all four samples are true clonally mutated genes, then proceed to step B), and B) administering to said mammal a therapeutically effective amount of a compound targeting said cancer cells containing at least one said true clonally mutated genes for treatment of said cancer cells. In one embodiment, wherein when the number of mutated genes in common with all four samples is less than the number of mutated genes in common between said three sample combinations then generating whole exome sequencing data, from an additional sample of cancer cells obtained from different regions in said mammal, comparing said sequencing data to corresponding wild-type reference genes in a databank, and thereafter: iii) Identifying the mutated genes in common between all five samples and identifying mutated genes that are in common between any four sample combinations, iv) determining whether said mutated genes in common between all five samples are the same as the mutated genes in common between said four sample combinations, wherein when the number of mutated genes in common with all five samples is equal to or greater than the number of mutated genes in common between said four sample combinations then mutated genes in common in all five samples are true clonally mutated genes, then proceed to step B).

Thus, in one embodiment, the invention provides a method for identifying a true clonal mutation in cancer cells from a mammal for use in treating said mammal, comprising, A) generating whole exome sequencing data, from at least five samples of cancer cells obtained from different regions in said mammal, comparing said sequencing data to corresponding wild-type reference genes in a databank, and thereafter: i) Identifying the mutated genes in common between all five samples and identifying mutated genes that are in common between any four sample combinations, ii) determining whether said mutated genes in common between all five samples are the same as the mutated genes in common between said four sample combinations, wherein when the number of mutated genes in common with all five samples is equal to or greater than the number of mutated genes in common between said four sample combinations then mutated genes in common in all five samples are true clonally mutated genes, then proceed to step B), and B) administering to said mammal a therapeutically effective amount of a compound targeting said cancer cells containing at least one said true clonally mutated genes for treatment of said cancer cells. In one embodiment, wherein when the number of mutated genes in common with all five samples is less than the number of mutated genes in common between said four sample combinations then generating whole exome sequencing data, from an additional sample of cancer cells obtained from different regions in said mammal, comparing said sequencing data to corresponding wild-type reference genes in a databank, and thereafter: iii) Identifying the mutated genes in common between all six samples and identifying mutated genes that are in common between any five sample combinations, iv) determining whether said mutated genes in common between all six samples are the same as the mutated genes in common between said five sample combinations, wherein when the number of mutated genes in common with all six samples is equal to or greater than the number of mutated genes in common between said five sample combinations then mutated genes in common in all six samples are true clonally mutated genes, then proceed to step B).

Thus, in one embodiment, the invention provides a method for identifying a true clonal mutation in cancer cells from a mammal for use in treating said mammal, comprising, A)

generating whole exome sequencing data, from at least six samples of cancer cells obtained from different regions in said mammal, comparing said sequencing data to corresponding wild-type reference genes in a databank, and thereafter: i) Identifying the mutated genes in common between all six samples and identifying mutated genes that are in common between any five sample combinations, ii) determining whether said mutated genes in common between all six samples are the same as the mutated genes in common between said five sample combinations, wherein when the number of mutated genes in common with all six samples is equal to or greater than the number of mutated genes in common between said five sample combinations then mutated genes in common in all six samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the invention provides a method for identifying a true clonal mutation in cancer cells from a mammal for use in treating said mammal, comprising, A)

generating whole exome sequencing data, from at least seven samples of cancer cells obtained from different regions in said mammal, comparing said sequencing data to corresponding wild-type reference genes in a databank, and thereafter: i) Identifying the mutated genes in common between all seven samples and identifying mutated genes that are in common between any six sample combinations, ii) determining whether said mutated genes in common between all seven samples are the same as the mutated genes in common between said six sample combinations, wherein when the number of mutated genes in common with all seven samples is equal to or greater than the number of mutated genes in common between said six sample combinations then mutated genes in common in all seven samples are true clonally mutated genes, then proceed to step B), and B) administering to said mammal a therapeutically effective amount of a compound targeting said cancer cells containing at least one said true clonally mutated genes for treatment of said cancer cells. Thus, in one embodiment, the invention provides a method for identifying a true clonal mutation in cancer cells from a mammal for use in treating said mammal, comprising, A) generating whole exome sequencing data, from at least eight samples of cancer cells obtained from different regions in said mammal, comparing said sequencing data to corresponding wild-type reference genes in a databank, and thereafter: i) Identifying the mutated genes in common between all eight samples and identifying mutated genes that are in common between any seven sample combinations, ii) determining whether said mutated genes in common between all eight samples are the same as the mutated genes in common between said seven sample combinations, wherein when the number of mutated genes in common with all eight samples is equal to or greater than the number of mutated genes in common between said seven sample combinations then mutated genes in common in all eight samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the invention provides a method for identifying a true clonal mutation in cancer cells from a mammal for use in treating said mammal, comprising, A) generating whole exome sequencing data, from at least nine samples of cancer cells obtained from different regions in said mammal, comparing said sequencing data to corresponding wild-type reference genes in a databank, and thereafter: i) Identifying the mutated genes in common between all nine samples and identifying mutated genes that are in common between any eight sample combinations, ii) determining whether said mutated genes in common between all nine samples are the same as the mutated genes in common between said eight sample combinations, wherein when the number of mutated genes in common with all nine samples is equal to or greater than the number of mutated genes in common between said eight sample combinations then mutated genes in common in all nine samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the invention provides a method for identifying a true clonal mutation in cancer cells from a mammal for use in treating said mammal, comprising, A) generating whole exome sequencing data, from at least ten samples of cancer cells obtained from different regions in said mammal, comparing said sequencing data to corresponding wild-type reference genes in a databank, and thereafter: i) Identifying the mutated genes in common between all ten samples and identifying mutated genes that are in common between any nine sample combinations, ii) determining whether said mutated genes in common between all ten samples are the same as the mutated genes in common between said nine sample combinations, wherein when the number of mutated genes in common with all ten samples is equal to or greater than the number of mutated genes in common between said nine sample combinations then mutated genes in common in all ten samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the Invention provides a method for identifying a true clonal mutation in cancer cells from a mammal for use in treating said mammal, comprising, A) generating whole exome sequencing data, from at least eleven samples of cancer cells obtained from different regions in said mammal, comparing said sequencing data to corresponding wild-type reference genes in a databank, and thereafter: i) Identifying the mutated genes in common between all eleven samples and identifying mutated genes that are in common between any ten sample combinations, ii) determining whether said mutated genes in common between all eleven samples are the same as the mutated genes in common between said ten sample combinations, wherein when the number of mutated genes in common with all eleven samples is equal to or greater than the number of mutated genes in common between said ten sample combinations then mutated genes in common in all eleven samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the invention provides a method for identifying a true clonal mutation in cancer cells from a mammal for use in treating said mammal, comprising, A) generating whole exome sequencing data, from at least twelve samples of cancer cells obtained from different regions in said mammal, comparing said sequencing data to corresponding wild-type reference genes in a databank, and thereafter: i) Identifying the mutated genes in common between all twelve samples and identifying mutated genes that are in common between any eleven sample combinations, ii) determining whether said mutated genes in common between all twelve samples are the same as the mutated genes in common between said eleven sample combinations, wherein when the number of mutated genes in common with all twelve samples is equal to or greater than the number of mutated genes in common between said eleven sample combinations then mutated genes in common in all twelve samples are true clonally mutated genes, then proceed to step B), and B) administering to said mammal a therapeutically effective amount of a compound targeting said cancer cells containing at least one said true clonally mutated genes for treatment of said cancer cells.

Thus, in one embodiment, the invention provides a method for treating cancer in a mammalian subject in need thereof, comprising, A) identifying a mutation in said cancer as a clonal mutation, wherein said identifying comprises, i) collecting n independent samples from said cancer, wherein n is an integer number greater than 1, ii) sequencing the n samples and determining all clonal mutations in each sample independently, iii) taking the intersection of all mutations identified as clonal in all n cancer samples (such as by using one or more of the equations disclosed herein), iv) taking the intersection of all mutations identified as clonal for all combinations of 1 to n−1 cancer samples, and v) calculating the probability that the mutations identified in steps (iii) and (iv) coincide, wherein the calculating comprises one or more of the equations disclosed herein, and wherein a calculated probability of at least 95% (such as 95%, 96%, 97%, 98%, 99%, and/or 100%) for a mutation identifies said mutation as a clonal mutation, and B) administering to said subject a therapeutically effective amount of a compound that alters expression, e.g. the biological effect, of said clonal mutation, thereby treating said cancer. In one embodiment, the compound specifically alters expression of said clonal mutation.

In another embodiment, the invention provides a method for identifying a mutation in cancer as a clonal mutation, comprising, i) collecting n independent samples from said cancer, wherein n is an integer number greater than 1, ii) sequencing the n samples and determining all clonal mutations in each sample independently, iii) taking the intersection of all mutations identified as clonal in all n cancer samples (such as by using one or more of the equations disclosed herein), iv) taking the intersection of all mutations identified as clonal for all combinations of 1 to n−1 cancer samples (such as by using one or more of the equations disclosed herein), and v) calculating the probability that the mutations identified in steps (iii) and (iv) coincide, wherein the calculating comprises one or more of the equations disclosed herein, and wherein a calculated probability of at least 95% (such as 95%, 96%, 97%, 98%, 99%, and/or 100%) for a mutation identifies said mutation as a clonal mutation.

### BRIEF DESCRIPTION OF THE DRAWINGS

FIG. 1: Shown is the remaining uncertainty σ=fⁿ+(1−f)ⁿ (colours) to identify all clonal mutations with n independent tumor samples and different degrees of tree balance f. For a totally balanced tree f=0.5, only few independent samples are necessary to achieve a high confidence. In contrast, if at least one very small clone is present f→0, the gain of additional samples becomes incremental and the uncertainty remains high.

FIG. 2: Shown are two examples of multi-region sequenced clear cell renal carcinoma. The data was taken from (17). a,b The number of clonal mutations for all possible combinations of sub-samples. a In total 11 samples were available, whereas in b the exome of 8 samples were sequenced. The bars correspond to the maximal and minimal number of identified clonal mutations per sample combination, whereas the boxes represent the 75% confidence intervals. In a only 6 coding mutations are clonal across all 11 samples, whereas in b 25 coding mutations are clonal across all 8 samples. c,d We calculate the probability (dots) to identify all clonal mutations in a and b respectively using the algorithm (i)-(v). We then fitted Equation (7) (lines) to the data to get an estimate for the fraction f of the left and right side of the tumor. In patient c we find a perfectly balanced size f=0.5 suggesting a neutrally expanding tumor, whereas in d the sides are highly unbalanced f=0.01 and fitness differential between subclones are likely. Interestingly, the neutral case was untreated before sampling and was 1 out of 2 cases that presented without metastatic disease, whereas the unbalanced case was treated with the mTOR inhibitor everolimus and developed metastasis. For a summary of all 10 cases, see FIGS. 3 and 4.

FIG. 3: Total number of clonal mutations identified in 10 patients with clear cell renal carcinoma and variable number of tumor samples. The data was taken from (17). All patients but RMH008 and RK26 developed metastasis. Patients RMH002, RMH008 and RK26 are treatment naïve. In general, a low number of tumor samples results in a significant variability in the number of identified clonal mutations. In all cases, the number of clonal mutations declines with increasing number of tumor samples. In some cases, the full information is already reached by a smaller set of tumor samples (for example, 5 samples in patient RK26) and additional samples do not provide further information.

FIG. 4: Fit of Equation (7) to the same set of patients presented in FIG. 3. Dots represent the probability to find the minimal set of clonal mutations according to the algorithm (i)-(v). Lines correspond to best fits of Equation (7). In 2 cases (EV003 and RK26) we find a balanced left and right side (f=½), suggesting a neutrally growing tumor. One case (RMH008) presents between a balanced and unbalanced case (f=0.32). This is potentially due to convergent evolution. All other cases are highly unbalanced (f<0.1), potentially indicating on going clonal selection.

FIG. 5: The sampling bias of a multi region analysis depends on a tumor's evolutionary history. FIG. 5a and FIG. 5b: The most recent common ancestor of all cells in the tumor contains all alterations that are truly clonal (top square). The first bifurcation from the ancestor divides the tumor into two populations that will constitute a fraction of f and 1−f at diagnosis. These fractions are the result of complex processes (e.g. clonal selection) and tumors might be balanced (both populations reach a similar size, f=0.5), or one population gains a significant fitness advantage and the tumor becomes unbalanced (f<<0.5). During growth, cells accumulate further alterations that contribute to intra tumor heterogeneity at diagnosis. FIG. 5c: This implies that different multi-region samples will identify different alterations and different combinations of samples will identify different sets of clonal and sub-clonal alterations. Only if we sample cells from both sides of the phylogenetic tree, we can identify all true clonal alterations. FIG. 5d: The probability that at least one out of i samples is from each side of the phylogenetic tree depends on the relative sizes of both sides f and is given by pf=1−fi−(1−f)i. Balanced trees (f=0.5) need few samples to identify all true clonal mutations with high confidence, while unbalanced trees (e.g. f=0.166) require more samples for the same confidence.

FIG. 6: Exemplary charts showing information gain from multi-region sequencing in patients with clear cell renal carcinoma. These charts include the information shown in FIGS. 2-4 along with additional data. FIG. 6 Panels a1 to j1: If from a set of n multi-region samples from a patient we consider different subsets of samples (n is between 5 and 11 per patient) with size i=1, 2, . . . n, we will identify different numbers of putatively clonal alterations, with great variation between different sets of the same size. The more samples we consider, the closer we get to the minimal identifiable set of clonal mutations, i.e. mutations that may have appeared clonal with one or few samples, turn out to be indeed sub-clonal in the whole tumor. FIG. 6 Panels a2 to j2: The probability to find the minimal set of clonal mutations falls onto the universal curve (8). Dots represent the data; lines correspond to best fits of f via Equation (8). In 2 cases (c2 and j2) we find a balanced left and right side (f=0.5). One case (i) appears slightly unbalanced (f=0.32) while all other cases are unbalanced (f<0.01), supporting the presence of convergent evolution and on-going clonal selection. All patients but (i2) and (j2) developed metastasis. Only patients (h2 to j2) are treatment naïve. For balanced tumors, the information on the true set of clonal alterations quickly plateaus with few samples (for example 5 samples in patient (j)). FIG. 6 Panels a3 to j3: We repeat the inference of the balancing factor f on all available combinations of subsets of tumor samples with a minimum of 4 samples. The violin plots show the corresponding distributions of f values for each possible combination of i=4, 5, . . . n−1 subsets. Most combinations of samples resemble the balancing inferred from the full data set. However, there is a trend towards a bimodal distribution for small i, which might be a direct consequence of the spatial evolution of tumors. Note that violin plots show the probability density distribution of the f-values. The actual f-values are never negative. Data from Gerlinger, M. et al. Genomic architecture and evolution of clear cell renal cell carcinomas defined by multiregion sequencing. Nature Genetics 46, 225-233 (2014).

FIG. 7: Exemplary charts showing information gain from multi-region copy number profiling in patients with colorectal cancer. Copy number changes were inferred from spatially distributed single glands of 11 colorectal tumors. Based on the shape of the universal curve (Equation (7)), 7 tumors appear balanced or nearly balanced and 4 tumors appear unbalanced. Balanced tumors require fewer samples to identify truly clonal copy number changes, whereas uncertainty remains high in unbalanced trees. Data from Sottoriva A, et al. (2015) A Big Bang model of human colorectal tumor growth. Nature Genetics. doi:10.1038/ng.3214.

### DEFINITIONS

To facilitate understanding of the invention, a number of terms are defined below.

“Treating” disease (e.g., cancer) refers to delaying, reducing, palliating, ameliorating, stabilizing, preventing and/or reversing one or more symptoms (such as objective, subjective, pathological, clinical, sub-clinical, etc.) of the disease. Objective symptoms are exemplified by tumor size (e.g. dimensions, weight and/or volume), tumor number, rate of change in tumor size and/or number, presence of metastasis, metastasis size (e.g. dimensions, weight and/or volume), metastasis number, and/or rate of change in metastasis size and/or number.

Subjective symptoms are exemplified by pain, fatigue, etc. Cancer symptoms may be assessed by, for example, biopsy and histology, and blood tests to determine relevant enzyme levels or circulating antigen or antibody, and imaging tests which can be used to detect a decrease in the growth rate or size of a neoplasm.

The terms “therapeutic amount,” “pharmaceutically effective amount,” and “therapeutically effective amount,” are used interchangeably herein to refer to an amount that is sufficient to achieve a desired result, such as treating disease. It need not cure the disease.

“Cancer” refers to a plurality of cancer cells that may or may not be metastatic, such as ovarian cancer, breast cancer, lung cancer, prostate cancer, cervical cancer, pancreatic cancer, colon cancer, stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, multiple myeloma and leukemia.

“Cancer cell” refers to a cell undergoing early, intermediate or advanced stages of multi-step neoplastic progression as previously described (Pitot et al., Fundamentals of Oncology, 15-28 (1978)). This includes cells in early, intermediate and advanced stages of neoplastic progression including “pre-neoplastic cells (i.e., “hyperplastic cells and dysplastic cells), neoplastic cells in advanced stages of neoplastic progression of a dysplastic cell, and metastatic cancer cells.

“Metastatic” cancer cell refers to a cancer cell that is translocated from a primary cancer site (i.e., a location where the cancer cell initially formed from a normal, hyperplastic or dysplastic cell) to a site other than the primary site, where the translocated cancer cell lodges and proliferates.

“Mammal” includes a human, non-human primate, murine (e.g., mouse, rat, guinea pig, hamster), ovine, bovine, ruminant, lagomorph, porcine, caprine, equine, canine, feline, ave, etc. In one preferred embodiment, the mammal is murine. In another preferred embodiment, the mammal is human.

A subject “in need” of treatment with the invention's methods and/or compositions includes a subject that is “suffering” from disease (i.e., a subject that is experiencing and/or exhibiting one or more symptoms of the disease), and subject “at risk” of the disease. A subject “in need” of treatment includes animal models of the disease. Subject “at risk” of disease refers to a subject that is not currently exhibiting disease symptoms and is predisposed to expressing one or more symptoms of the disease. This predisposition may be based on family history, genetic factors, environmental factors such as exposure to detrimental compounds present in the environment, etc.). It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

The terms “mutation” and “modification” refer to a deletion, insertion, or substitution. A “deletion” is defined as a change in a nucleic acid sequence or amino acid sequence in which one or more nucleotides or amino acids, respectively, is absent. An “insertion” or “addition” is that change in a nucleic acid sequence or amino acid sequence that has resulted in the addition of one or more nucleotides or amino acids, respectively. A “substitution” in a nucleic acid sequence or an amino acid sequence results from the replacement of one or more nucleotides or amino acids, respectively, by a molecule that is a different molecule from the replaced one or more nucleotides or amino acids. For example, a nucleic acid may be replaced by a different nucleic acid as exemplified by replacement of a thymine by a cytosine, adenine, guanine, or uridine. Alternatively, a nucleic acid may be replaced by a modified nucleic acid as exemplified by replacement of a thymine by thymine glycol. Substitution of an amino acid may be conservative or non-conservative. “Conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains that may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. “Non-conservative substitution” is a substitution other than a conservative substitution. Guidance in determining which and how many amino acid residues may be substituted, inserted or deleted without abolishing biological and/or immunological activity may be found using computer programs well known in the art, for example, DNAStar™ software.

A “clone” as used herein refers to a population of cells that is descended from a single common ancestor cell and that contains the same genetic mutations. Thus, the term “clonal mutation” when made in reference to a cell population (e.g., cancer cell population) refers to a mutation that is present in each and every cell in the population.

A “sample” when in reference to cancer includes, without limitation, cells (such as cell lines, cells isolated from tissue whether or not the isolated cells are cultured after isolation from tissue, fixed cells such as cells fixed for histological and/or immunohistochemical analysis), tissue (such as biopsy material), cell extract, tissue extract, amino acid sequence, and/or nucleic acid sequence (e.g., DNA and RNA), that are obtained from a subject, including body fluids (such as urine, blood, plasma, fecal matter, cerebrospinal fluid (CSF), semen, sputum, and saliva), as well as solid tissue. In one embodiment, samples may be obtained from different regions of the cancer tissue and/or from the same cancer tissue over a period of time.

The term “administering” to a subject means delivering one or more compounds (e.g., therapeutic drug) to a subject, including prophylactic administration of the composition (i.e., before disease and/or one or more symptoms of disease are detectable) and/or therapeutic administration of the composition (i.e., during and/or after the disease and/or one or more symptoms of the disease are detectable). Administering may be done using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789), hereby incorporated by reference. Also, the invention's compositions may be administered before, concomitantly with, and/or after administration of another type of drug or therapeutic procedure (e.g., surgery). Administration may be parenteral (e.g., subcutaneous, intravenous, intramuscular, intrastemal injection, and by infusion), oral, intraperitoneal, intranasal, topical, sublingual, etc.

The terms “reduce,” “inhibit,” “diminish,” “suppress,” “decrease,” and grammatical equivalents (including “lower,” “smaller,” etc.) when in reference to the level of any molecule (e.g., therapeutic compound, amino acid sequence, nucleic acid sequence, antibody, etc.), cell, and/or phenomenon (e.g., level of expression of a clonal mutation, expression of a gene, disease symptom, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than (including at least one of the following: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) lower than the quantity of the same molecule, cell and/or phenomenon in the second sample (or in the second subject).

The terms “increase,” “elevate,” “raise,” and grammatical equivalents (including “higher,” “greater,” etc.) when in reference to the level of any molecule (e.g., therapeutic compound, amino acid sequence, nucleic acid sequence, antibody, etc.), cell, and/or phenomenon (e.g., level of expression of a clonal mutation, expression of a gene, disease symptom, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than (including at least one of the following: 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) greater than the quantity of the same molecule, cell and/or phenomenon in the second sample (or in the second subject).

The terms “alter” and “modify” when in reference to the level of any molecule (e.g., therapeutic compound, amino acid sequence, nucleic acid sequence, antibody, etc.), cell, and/or phenomenon (e.g., level of expression of a clonal mutation, expression of a gene, disease symptom, etc.) in a first sample (or in a first subject) relative to a second sample (or relative to a second subject), mean an increase and/or decrease.

### DESCRIPTION OF THE INVENTION

The identification of true clonal mutations in a patient's tumor is key for current approaches of targeted therapies. Targeted therapies are doomed to fail without the presence of the actual target. Here we show that a single tumor sample is unlikely to identify true clonal mutations and multiple samples are required. The actual number of samples depends on the evolutionary history of the tumor. Neutrally expanding tumors require only few samples, 8 samples provide a confidence of 99% to identify all true clonal mutations. Non-neutral tumors with on-going clonal selection require more samples, whereas the number depends on the relative size of clones.

I. Treating Cancer Cells Having True Clonal Mutations.

The first successful branching of the initial ancestor cell determines all true clonal mutations. All subsequent branching events introduce mutations that remain sub-clonal and thus are not amongst the true clonal mutations. This also suggests that small but late occurring sub-clones do not change our conclusion. They might have important implications for the future evolution of the tumor, such as the risk of metastatic spread or the occurrence of resistance, but they don't influence the composition of the true set of clonal mutations.

It is long known that Intra tumor heterogeneity is a major obstacle of cancer treatment and much of our failures to cure cancer are contributed to it (Sawyers, C. L. The cancer biomarker problem. Nature 452, 548-552 (2008); 11-13). Data herein shows that reasonable targets for therapy must be amongst the true clonal mutations that are present in all cancerous cells. This information cannot be gained from single tumor samples. We provide a rational of how many samples are necessary for a certain confidence to identify these clonal mutations. In many cases, these numbers are surprisingly low. They are already reached in first pilot studies and can be realistically introduced in diagnostic protocols in the near future.

Thus, in one embodiment, the invention provides a method for treating cancer in a mammalian subject in need thereof, comprising, A) identifying a mutation in said cancer as a clonal mutation, wherein said identifying comprises, i) collecting n independent samples from said cancer, wherein n is an integer number greater than 1, (including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, etc.)), ii) sequencing the n samples and determining all clonal mutations in each sample independently, iii) taking the intersection of all mutations identified as clonal in all n cancer samples (such as by using one or more of the equations disclosed herein), iv) taking the intersection of all mutations identified as clonal for all combinations of 1 to n−1 cancer samples (such as by using one or more of the equations disclosed herein), and v) calculating the probability that the mutations identified in steps (iii) and (iv) coincide, wherein the calculating comprises one or more of the equations disclosed herein, and wherein a calculated probability of at least 95% (such as 95%, 96%, 97%, 98%, 99%, and/or 100%) for a mutation identifies said mutation as a clonal mutation, and B) administering to said subject a therapeutically effective amount of a compound that alters expression of said clonal mutation, thereby treating said cancer. In a preferred embodiment, the compound specifically alters expression of the clonal mutation. See examples of specific treatments below.

II. Identifying Cancer Cells Having True Clonal Mutations.

In another embodiment, the invention provides a method for identifying a mutation in cancer as a clonal mutation, comprising, i) collecting n independent samples from said cancer, wherein n is an integer number greater than 1 (including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, etc.)), ii) sequencing the n samples and determining all clonal mutations in each sample independently, iii) taking the intersection of all mutations identified as clonal in all n cancer samples (such as by using one or more of the equations disclosed herein), iv) taking the intersection of all mutations identified as clonal for all combinations of 1 to n−1 cancer samples (such as by using one or more of the equations disclosed herein), and v) calculating the probability that the mutations identified in steps (iii) and (iv) coincide, wherein the calculating comprises one or more of the equations disclosed herein, and wherein a calculated probability of at least 95% (such as 95%, 96%, 97%, 98%, 99%, and/or 100%) for a mutation identifies said mutation as a clonal mutation.

III. Treating Cancer Cells in Patients: Guiding Targeted Therapy.

Targeted therapy refers to drug treatments that targets the cancer's specific genes, proteins, or the tissue environment that contributes to cancer growth and survival. These genes and proteins are found in cancer cells, e.g. genes and proteins inducing cells to divide abnormally or live too long. In some cases, targeted genes are expressed in cells related to cancer growth, e.g. blood vessel cells. Therefore targeted therapy treatment blocks or helps to slow the growth and spread of cancer cells while limiting damage to healthy cells. Thus, in preferred embodiments, drugs (compounds) may block or turn off the signals that tell cancer cells to grow and divide; may keep cells from living longer than normal, or may kill the cancer cells. Nonlimiting examples of compounds include monoclonal antibodies; “small-molecule drugs, etc.

The challenges of target therapy include findings that not all of the same type of tumors have the same targets. So the same targeted treatment does not work for everyone.

As one example, in some metastatic Clear Cell Renal Cell Carcinoma (ccRCC) patents, with PBRM1 (Protein polybromo-1 (PB1)) mutations, these patients exhibited longer median first-line progression free survival (PFS1L) with first-line Everolimus (an inhibitor of ‘mammalian target of rapamycin’: mTOR) treatment but not with first line Sunitinib (multitargeted tyrosine kinase inhibitor) treatments. See, Hsieh, et al., Genomic Biomarkers of a Randomized Trial Comparing First-line Everolimus and Sunitinib in Patients with Metastatic Renal Cell Carcinoma. European Urology, Volume 71, Issue 3, Pages 405-414, 2017.

However, because targeted therapy may not be effective enough to slow cancer cell growth and/or spread, patients' may also need surgery, chemotherapy, radiation therapy, or hormone therapy. Targeted therapies may also be used as adjuvant therapies, which are treatments given after the main treatment(s) (first line) to lower the risk of recurrence and to remove remaining cancer cells.

According to their significance, mutations in cancer cells were classified into drivers and passengers. Driver mutations include, but are not limited to, those implicated in tumor initiation and progression. Compounds that specifically alter expression of mutations are known in the art. For example, certain tumors contain activating mutations in driver genes encoding protein kinases. This has led to the development of small-molecule inhibitor drugs targeting those kinases. Representative examples of this type of genome-based medicine include the use of EGFR kinase inhibitors to treat cancers with EGFR gene mutations (Sharma S V, et al. Nat Rev Cancer. 2007; 7:169), the anaplastic lymphoma kinase (ALK) inhibitors to treat cancers with ALK gene translocations (25), and specific inhibitors of mutant BRAF to treat cancers with BRAF mutations (24).

Specific examples of cancers and some non-limiting examples of related mutations for colorectal cancer, breast cancer, lung cancer, and melanoma are described below that may find use in guiding targeted treatment when the associated mutation is identified as a true clonal mutation as described herein.

Colorectal cancers are often associated with mutations in epidermal growth factor receptor (EGFR), e.g. causing overexpression, etc. Drugs that block EGFR may help stop or slow cancer growth.

Lung cancer cells may have EGFR mutations and/or may have mutations in the ALK gene, such as ALK gene translocations treated with inhibitors as referenced above. In fact, the drug erlotinib (Tarceva®), which can be used to treat non-small cell lung cancer, works better in patients whose cancer cells have a certain mutation in the EGFR gene.

Breast cancer cells may have mutations resulting in overexpression of human epidermal growth factor receptor 2 (HER2) inducing tumor cell growth such that HER2 positive targeted therapies are used. In other cases, breast cancer cells may be HER2 negative indicating a different type of treatment. Melanoma cells frequently have mutations in the BRAF gene, thus specific BRAF mutations are drug targets. However, some of these drugs will increase melanoma cell growth when BRAF is not mutated. Thus, identification of BRAF as a true clonal mutation may increase the safety and efficacy of inhibitors of mutant BRAF as referenced above.

However, even though a patient's cancer cells may have such mutations, e.g. determined by results of genetic testing for familial kinase gene mutations in germline cells, or testing the actual cancer cells for a mutation, or identifying the mutation by watching to see which type of cancer treatment compound is most effective in reducing cancer cells in that patient, etc., a mutation may not be a true clonal mutation and/or a mutation directly related to growing and possibly spreading of cancer cells. Such that even though a patient's cancer has a gene mutation associated with the use of a particular class of drugs, such as when using EGFR kinase inhibitors to treat cancers having EGFR gene mutations, that patient may not always respond favorably to such treatment. For example, having a germline mutation in a gene related to susceptibly, such as EGFR, may not guarantee that an EGFR mutation is the true clonal gene mutation directly related to the growing cancer cells that should be the target of effective therapy.

Thus, in preferred embodiments, identification of a true clonal gene mutation is contemplated for use in directing cancer treatment in a patient. In some embodiments, identifying a true clonal gene mutation directs treatment of that cancer patient by guiding medical personal to use a drug (therapeutic compound) treatment where that drug is associated with positive responses in patients having that particular gene mutation. In preferred embodiments, such guided use would result in slowing cancer cell replication (in vitro and/or in vivo), indicating that its use would reduce cancer cells in a patient.

In contrast, many mutations contraindicate a particular drug treatment. Thus in some embodiments, identifying a true clonal gene mutation directs treatment of the cancer by avoiding choosing a drug associated with negative responses, e.g. the drug has no effect on or it increases cancer cell replication (in vitro and/or in vivo), such as when used to treat a patient.

In some further embodiments, identifying a true clonal gene mutation and identifying whether an associated gene is mutated directs treatment of the cancer. For example, colorectal cancer is associated with activation of the EGFR1 pathway, thus EGFR1 protein kinase inhibition is used for treatment. However, about 40% of colorectal cancers have a gene mutation in KRAS (Ki-ras2 Kirsten rat sarcoma viral oncogene homolog) that controls cancer cell growth and spread. In colorectal cancer, when targeted therapies such as cetuximab (Erbitux) and panitumumab (Vectibix), anti-EGFR1 therapeutic monoclonal antibodies are used for cancer cells also having activating KRAS mutations, patients have shorter survival rates. Thus, guided therapy for administering anti-EGFR1 therapeutic monoclonal antibodies is indicated for colorectal cancer patients without coexisting KRAS mutations.

Thus, a targeted treatment may not work when the cancer cells/tumor does not have the target mutation and conversely having the target mutation does not mean the cancer cells/tumor will respond to the drug. Therefore, testing for true clonal mutations is contemplated for use in identifying a target more medically relevant to the cancer cells to increase the probability that a patient's cancer cells/tumor will be directly and effectively guided as described herein.

A. Treating Kidney Cancers: e.g. Clear Cell Renal Carcinoma (ccRCC).

Kidney tumors are estimated to be diagnosed in more than 270,000 individuals every year worldwide.1 More than 65,000 new diagnoses and approximately 13,680 patient deaths as a result of tumors of the kidney and renal pelvis were projected in the United States for 2013. Primary Kidney cancer cells arise when healthy cells in 1 or both kidneys change and grow out of control, forming a mass referred to as a renal cortical tumor. A tumor can be malignant, indolent, or benign. A malignant tumor is cancerous, meaning it can grow and spread to other parts of the body. An indolent tumor is also cancerous, but this type of tumor rarely spreads to other parts of the body. A benign tumor means the tumor can grow but will not spread. There are numerous types of kidney cancers, including but not limited to Renal Cell Carcinoma; transitional cell carcinoma (urothelial carcinoma); Wilms tumor; Kidney Lymphoma; Kidney Sarcoma; etc.

Types of kidney cancer cells in any of these types of tumors include clear cell; chromophobe; papillary (at least 2 different subtypes, called type 1 and type 2); transitional cell carcinoma; medullary/collecting duct tumor, related to transitional cell carcinoma, highly associated with having the sickle cell trait. Each of the tumor subtypes of clear cell, chromophobe, and papillary in kidney cancer can show highly disorganized features under the microscope. These are often described by pathologists as “sarcomatoid.” This may not be a distinct tumor subtype, but when these features are seen it is associated with an aggressive form of kidney cancer.

Papillary kidney cancer is currently treated in the same way as clear cell kidney cancer. However treatment with targeted therapy is often not as successful for people with papillary kidney cancer as it is for people with clear cell kidney cancer. Thus, in one embodiment, determining a true clonal mutated gene in clear cell kidney cancer may be used to guide therapy e.g. a therapy related to a true clonal mutation. In another embodiment, determining a true clonal mutated gene in papillary kidney cancer may be used to guide therapy, e.g. a therapy related to a true clonal mutation.

Clear cell kidney cancer may have a mutation of the von Hippel-Lindau (VHL) gene (typically inactivated by either mutation or methylation in over 80% of ccRCC), or a mutation in an associated gene, such as an Elongin C gene (i.e. TCEB1), causing the cancer to make too much of vascular endothelial growth factor (VEGF) protein. VEGF controls the formation of new blood vessels. Thus, when a true clonal mutation in a VHL gene is identified then TKI drugs may be administered to help block VEGF and other chemical signals that promote the development of new blood vessels. Examples of TKIs include cabozantinib (Cabometyx), pazopanib (Votrient), sorafenib (Nexavar), sunitinib (Sutent), bevacizumab (Avastin) (including for treating metastatic renal carcinoma where a VHL mutation is identified as a true clonal mutation), axitinib, temsirolimus, everolimus, etc., may be used for treating clear cell kidney cancer. In particular, TKIs may be indicated for VEGFR true clonal mutations while bevacizumab may be indicated for VEGF true clonal mutations. Another TKI, Axitinib (Inlyta) may be used to treat later-stage renal cell carcinoma where VHL is identified as a true clonal mutation. Further, such drugs may be combined with other therapies, for one example Bevacizumab may be combined with interferon (Immunotherapy) for slowing tumor growth and/or spread (i.e. metastasis).

mTOR kinase inhibitors may also be used, such as Everolimus (Afinitor) and temsirolimus (Torisel) that target mTOR kinase activity which helps kidney cancer cells grow, thus slowing kidney cancer growth. In such cases where mTOR is targeted, mTOR may not be the identified true clonal mutation such that a downstream mTOR effector regulating angiogenesis, metabolism, and/or cell growth may be identified as the true clonal mutated gene.

The BRCA1 associated protein-1 (BAP1) gene was also found mutated in 10% to 15% of patients with ccRCC. Thus in some embodiments, when a true clonally gene mutation is in BAP1, then a guided therapy targets BAP1. In other embodiments, a PBRM1 gene may be identified as a true clonally mutated gene for targeting with drug therapy.

In some embodiments, said true clonally mutated gene is considered a one-hit tumor suppressor gene, such that one mutation in one of the two alleles is required to drive cancer cell growth, one occurring in one of the two alleles of the same gene. In some embodiments, said true clonally mutated gene is considered a two-hit tumor suppressor gene, such that two mutations are required to drive cancer cell growth, one occurring in each of the two alleles of the same gene, e.g. VHL, BAP1, etc. Thus in some embodiments, said identification of said sequenced clonal gene further comprises determining whether said clonal mutation is a one-hit or two-hit mutation.

B. Treating Gastrointestinal Cancers: e.g. Colorectal Cancer (CRC).

Gastrointestinal Cancer refers to a group of cancers that affect the digestive system, including cancers (tumors) of the esophagus, gallbladder, liver, pancreas, stomach, small intestine, bowel (large intestine or colon and rectum), and anus.

For examples of mutations related to treatment of gastric cancers, when such cancers have high-level clonal FGFR2 amplification, i.e. FGFR2 mutation, these cancers have a high response rate to the selective FGFR inhibitor AZD4547, whereas cancers with subclonal or low-level amplification did not respond. Pearson, et al., “High-Level Clonal FGFR Amplification and Response to FGFR Inhibition in a Translational Clinical Trial.” Cancer Discov Aug. 1, 2016 (6)(8):838-851. In fact, Pearson et al, Cancer Discovery, (2016)) showed that the best response to FGFR inhibitor therapy occurred in patients whose tumors were homogenous (i.e. having few or no branches) for FGFR amplification. Non-responding tumors had sub-clonal heterogeneous FGFR amplification and the presence of FGFR non-amplified tumor cells. Thus, targeting the trunk or early clonal events is contemplated to increase the response to targeted therapy such that a greater proportion of cancer cells in the patient share the same genetic alterations. Further, Pearson et al, showed an overlap of mutations from two different metastatic sites biopsied from the same patient where the percentage of mutations shared between sites decreases after therapy. That is to say, there may be higher diversity in mutational profiles from different metastatic sites after treatment compared to untreated patients. Thus, in some embodiments, a mammal undergoing identification of true clonal mutations may be an untreated patient, such as a patient first diagnosed with cancer.

Further, some drugs don't help patients if the cancer cells have certain gene mutations. For example, cetuximab (Erbitux®) and panitumumab (Vectibix®) are drugs used to treat advanced colorectal cancers, including cancers with mutations in EGFR genes. However, these drugs don't help patients with cancers that also have mutations in the KRAS gene. Thus, mutational testing for genes in the EGFR signaling pathway may provide clinically actionable information as negative predictors of benefit to anti-EGFR monoclonal antibody therapies for targeted therapy of CRC. Sepulveda, et al., “Molecular Biomarkers for the Evaluation of Colorectal Cancer.” http://www.asco.org/practice-guidelines/quality-guidelines/guidelines/gastrointestinal-cancer#/15831. Feb. 6, 2017. and http://www.cancer.net/beyond-kras-testing-tumors-other-genetic-mutations-helps-personalize-treatment-metastatic-colorectal. Gastrointestinal Cancers Symposium Jan. 14, 2014.

In some embodiments, when true clonal mutations are identified in RCC, such as MET mutations, e.g. a germline or a somatic MET mutation, MET [7q31] amplification, or gain of chromosome 7, or a true clonal mutation in an associated gene, such as VEGF, RON, AXL, TIE-2 receptors, etc., then an oral multikinase inhibitor targeting MET, e.g. foretinib, is contemplated for guided treatment of patients having RCC, including in some cases for metastatic papillary RCC. Hass, et al., Hereditary Renal Cancer Syndromes. Adv Chronic Kidney Dis. 2014 21(1).

In summary, future personalized treatment strategies of human malignancies are contemplated to be based on information from multi-region profiling of tumours (Nicholas McGranahan et al. Clonal neoantigens elicit T cell immunoreactivity and sensitivity to immune checkpoint blockade. Science 351:1463-1469 (2016; 30). Once multi-region sampling becomes available in routine clinical practice, physicians will be able to make informed decisions on how many samples per tumor in the individual patient need to be independently sequenced for providing subsequent optimal therapy.

The study described herein, provides a rationale for how many samples are necessary to achieve a certain level of confidence that truly clonal alterations in a tumor have been identified from multi-region profiling. Assigning clonality to specific alterations implies also the identification of sub-clonal alterations. The distribution of sub-clonal alteration contains important information on the evolutionary history of tumours (Ling S, et al. (2015). Extremely high genetic diversity in a single tumor points to prevalence of non-Darwinian cell evolution. Proceedings of the National Academy of Sciences. doi:10.1073/pnas.1519556112; 27).

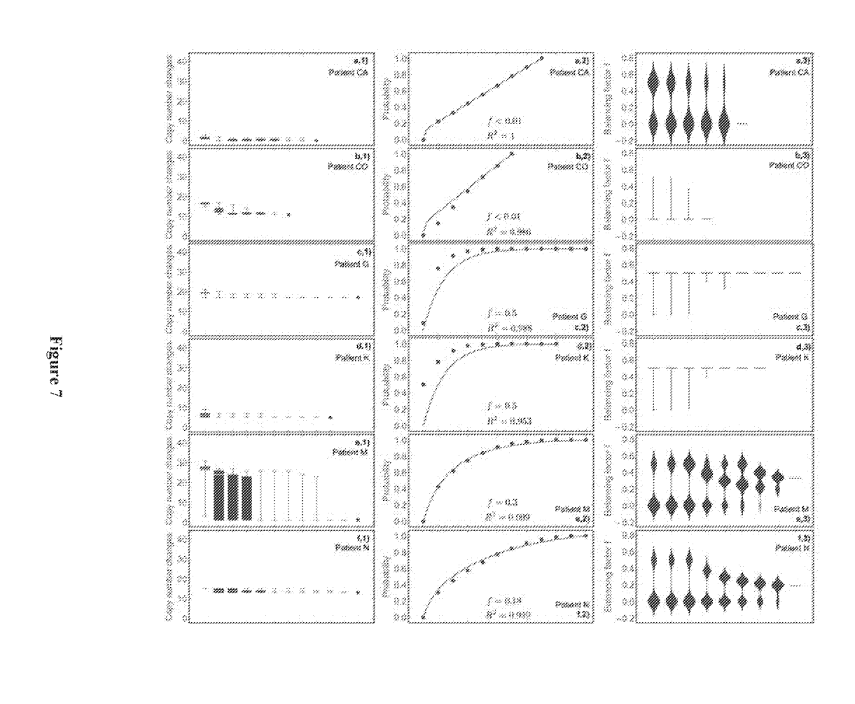

As one example, in FIG. 6, the floating bar charts on the left show the number of tumor samples taken from a patient vs. clonal mutations, i.e. where 1 sample is taken, vs. larger numbers of samples, the range of clonal mutations is larger than when 4 samples were taken. Correspondingly, the probability of identifying clonal mutations approaches 100% on a universal curve, i.e. equation (7), where reaching that level is different between patients, see charts in the middle column in FIG. 6. Balancing factor f (values) are calculated for the overall cancer, for each patient, from the data used in the left column, whose values are shown in the charts of the middle column. Further, when corresponding distributions of balancing factor f (values) are calculated for each possible combination of i=4, 5, . . . n−1 subsets, i.e. when each of the samples in the left column have 4 sub-samples, 5-sub-samples, etc., plotted on violin charts as shown in the right column, these charts show the probability density distribution of the f values corresponding to the number of i sub-samples for each sample n.

However, here we investigated the impact of standard multi-region profiling on treatment decision and focused on clonal alterations. Our method allows tailoring of the number of independent samples that is necessary for each individual tumor. Although the cost of genome sequencing is decreasing rapidly, the prospect of multiple sample profiling in each patient may present a new and daunting financial burden on healthcare systems, especially as the identification of truly clonal alterations in unbalanced tumors (f<<0.5) may be difficult and perhaps less cost-effective, posing new challenges. However, in many cases the required number of independently sequenced samples appears surprisingly manageable.

Our approach is independent of any threshold that is often imposed from a statistical analysis of the distribution of mutations identified in a tumor. Our analysis also suggests that the optimal time to perform genome profiling in tumors is at the time of diagnosis since therapy appears to introduce strong selection that may interfere with the identification of the therapeutically relevant truly clonal mutations or immune therapeutic targets (Nicholas McGranahan et al. Clonal neoantigens elicit T cell immunoreactivity and sensitivity to immune checkpoint blockade. Science 351:1463-1469 (2016); Gerlinger M, et al. (2012) Intratumor Heterogeneity and Branched Evolution Revealed by Multiregion Sequencing. New England Journal of Medicine 366(10):883-892). Tumors at relapse might require denser sampling compared to treatment naive tumors. The list of truly clonal mutations identified by our approach will potentially include tumor driver alterations that could be a targeted for therapy. Although our approach cannot identify a priori the driver mutations, this method will significantly restrict the search for such drivers in each particular patient and potentially identify targetable clonal mutations that are otherwise unknown. This study represents one of many necessary steps to advance from purely descriptive tumor sequencing towards individualized therapies based on quantitative evolutionary principles.

### EXPERIMENTAL

The following examples serve to illustrate certain preferred embodiments and aspects of the present invention and are not to be construed as limiting the scope thereof.

### Example 1

The Gain of Information by Multi-Region Sequencing

Let us consider the true phylogenetic tree of a tumor at a certain time t (e.g. at diagnosis). Each leaf of this tree is a cancer cell. Assume there are N leaves and therefore N−1 bifurcations in the tree. By definition, mutations present in the trunk of this tree are truly clonal and thus are in all cells of the tumor. The first bifurcation splits the tumor into two groups, the “left” side with proportion f, and the “right” side with proportion 1−f.

A. Cell Samples.

If we were to sample a single cell, many mutations carried by this cell would be likely not truncal. If we sampled a second cell we would increase our chance to detect the true clonal mutations. In general, we have three possibilities: with probability f² we have two cells from the left, with probability (1−f)² we have two cells from the right, and with probability 2f(1−f) we have one cell from the left and one from the right. In this last case, the mutations common to both cells are the true set of truncal mutations and consequently must be present in all cells of the tumor. We can determine the probability p to have picked cells from the left and from the right side of the tumor with n independent samples:

p_{f}(n)=1−fⁿ−(1−f)ⁿ (1)

resulting in a non-linear dependence of the probability to find the true set of clonal mutations on the number of samples n. A single sample never provides the full information, as p_{f}(1)=0 for any f>0. The expected gain of information with an additional sample n+1 is:

p_{f}(n+1)−p_{f}(n)=(1−f)ⁿf+(1−f)fⁿ. (2)

For example consider the case of a perfectly balanced tree (e.g. a neutrally expanding tumor (Williams M J, Werner B, Barnes C P, Graham T A, Sottoriva A (2016) Identification of neutral tumor evolution across cancer types. Nature Genetics 48:238-244)). This implies f=½ and the expected gain of information from sample n to sample n+1 is

$${p}_{f} ( n + 1 ) - {p}_{f} ( n ) = {( \frac{1}{2} )}^{n} . \qquad ( 3 )$$

The gain of additional samples decreases exponentially, in other words: in the case of balanced trees with f˜½, such as neutral or nearly-neutral trees, only few independent tumor samples are needed to identify all clonal mutations. If we define the remaining uncertainty to have missed the true clonal mutations σ=1−p, we can rearrange Equation (1) for the case of a balanced tree with f=½ and find the required number of samples n necessary for a certain confidence

n=1−log₂(σ). (4)

For example, a remaining uncertainty of 1% requires only n=8 independent tumor samples. This level of resolution is already reached in recent multi-region sequencing studies (14, 16, 18, 20) and poses a realistic target for daily clinical care in the near future.
However, one “side” of the tumor could be very small with f→0 (i.e. the tumor is highly unbalanced), implying that different parts of the tree have grown at radically different pace due to clonal selection. In this case, Equation (1) can be approximated by p_{f→0}(n)≈nf and the remaining uncertainty decreases linearly in n. For a sufficiently small n, the gain of information by an additional tumor sample becomes incremental

p_{f→0}(n+1)−p_{f→0}(n)≈f. (5)

Here many tumor samples are required to gain a high confidence of finding all true clonal mutations.

However, a very slowly growing side contributes very little, if at all, to the overall aggressiveness of the tumor, especially if this side virtually vanishes (f→+0). Although, many samples are needed to infer all true clonal alterations in this situation, the clonal alterations of the extremely dominant and tumor-driving side are of practical interest and again fewer samples may suffice. However, very small ancient sub-clones might drive tumor relapse, as is for example observed in certain leukaemias (31,32).

In general, the remaining uncertainty is given by

σ_{f}=fⁿ−(1−f)ⁿ, (6)

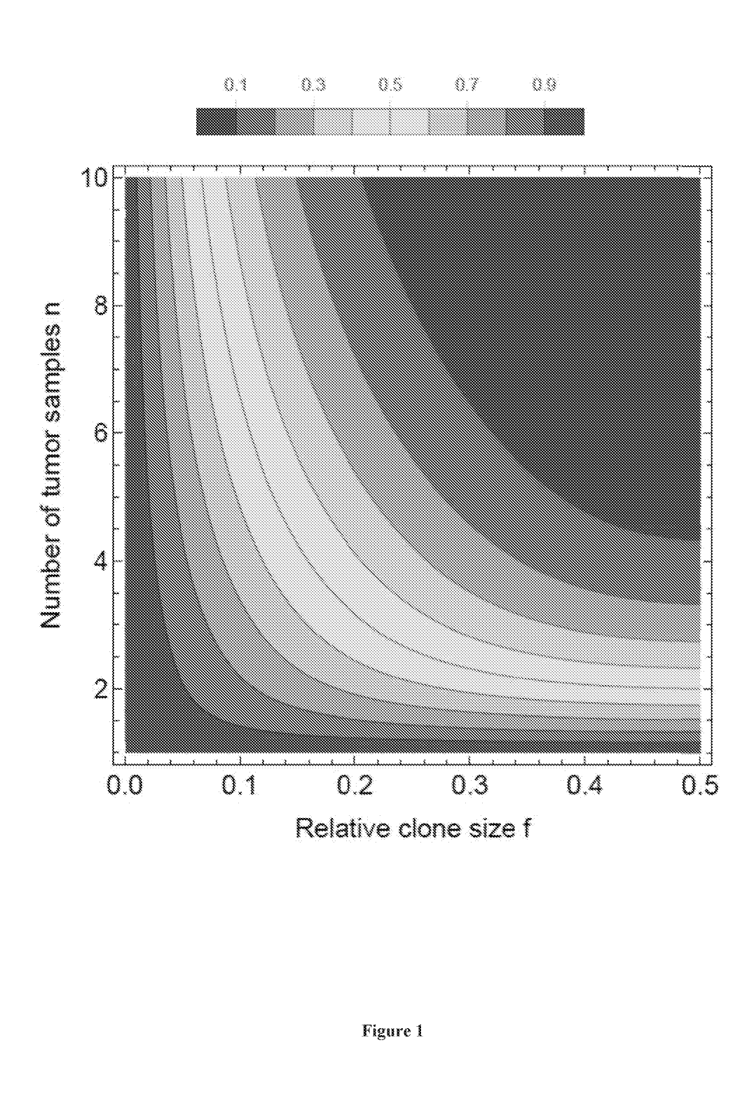

which lies between a linear (f→0) and an exponential (f→½) gain of confidence with additional samples n, see also FIG. 1.

B. Tissue Samples.

A calculation for tissues is similar for cells and uses the same equations (1-7).

If we were to take a single tissue sample, many alterations carried by this subpopulation would likely not be truncal. If we took a second tissue sample, we would increase our chance to identify truly clonal alterations. In this case, we have three possibilities: with probability f² we have two tissue samples from one side, with probability (1−f)² we have two tissue samples from the other side, and with probability 2f(1−f) we have one tissue sample from each side. Only in this last case, the alterations common to both samples would represent the true set of truncal (clonal) alterations and consequently must be present in all cells of the tumor. With n independent samples, the probability p to have picked both sides of the tumor becomes equation (1) resulting in a non-linear dependence of the probability to find the true set of clonal mutations through n samples.

A single sample never provides the full information, as p_{f}(1)=0 for n=1. The expected gain of information with an additional sample n+1 is equation (2).

For example consider the case of a perfectly balanced tree (e.g. a neutrally expanding tumor (27). This implies f=0.5 and the expected gain of information from sample n to sample n+1 is equation (3).

The information gain due to the inclusion of additional samples decreases exponentially, in other words: in the case of balanced trees with f is approximately 0.5, such as neutral or nearly-neutral trees, relatively few independent tumor samples are needed to identify all true clonal alterations. If we define the remaining uncertainty to have missed the true clonal alterations to be σ=1−p, we can rearrange Equation (1) for the case of a balanced tree with f=0.5 and find the required number of samples n necessary for a certain confidence expressed by equation (4).

For example, a remaining uncertainty of 1% requires only n≈8 independent tumour samples. This level of resolution has already been reached in several recent multi-region sequencing studies (Gerlinger M, et al. (2012) Intratumor Heterogeneity and Branched Evolution Revealed by Multiregion Sequencing. New England Journal of Medicine 366(10):883-892; Martincorena I, et al. (2015) High burden and pervasive positive selection of somatic mutations in normal human skin. Science 348:880-886; Sottoriva A, et al. (2015) A Big Bang model of human colorectal tumor growth. Nature Genetics. doi:10.1038/ng.3214.; Ling S, et al. (2015) Extremely high genetic diversity in a single tumor points to prevalence of non-Darwinian cell evolution. Proceedings of the National Academy of Sciences. doi:10.1073/pnas.1519556112.) and poses a realistic target for daily clinical care in the near future.

However, one “side” of the tumor could be very small with f<<0.5 (i.e. the tumor is highly unbalanced), implying that different parts of the tree have grown at radically different rates, e.g. due to clonal selection. In this case, Equation (1) can be approximated by p_{f→0}(n)≈nf and the remaining uncertainty decreases linearly in n. For sufficiently small n, the gain of information by an additional tumor sample becomes incremental as calculated by equation (5).

In this case, many tumor samples are required to reach a high level of confidence of finding all true clonal alterations. However, a very slowly growing side contributes very little, if at all, to the overall aggressiveness of the tumor, especially if this side virtually vanishes (f→0). Although, many samples are needed to infer all true clonal alterations in this situation, the clonal alterations of the extremely dominant and tumor-driving side are of practical interest and again fewer samples may suffice. However, very small ancient sub-clones might drive tumor relapse, as is for example observed in certain leukaemias (31,32).

In general, the remaining uncertainty is given by equation (6) which lies between a linear (f→0) and an exponential (f→½) gain of confidence with additional samples n.

C. Summary.

Let us consider the complete phylogenetic tree of a tumor. Each leaf of this tree is a cancer cell (or tissue). Leaves are separated by bifurcations representing cell divisions prone to inheritable alterations, which could be single nucleotide polymorphisms, gene duplications, translocations or any other genomic change. Alterations in cells may result in homogeneous or heterogeneous mutations within a tissue.

Alterations that are in the trunk of the tree must be present in all cells of the tumor or tissue, if we neglect unlikely events of back mutations. The first bifurcation divides the tumor into two populations of fraction f and 1−f. The sizes of these fractions are the result of potentially complicated processes, e.g. clonal selection, immune system escape or random drift.

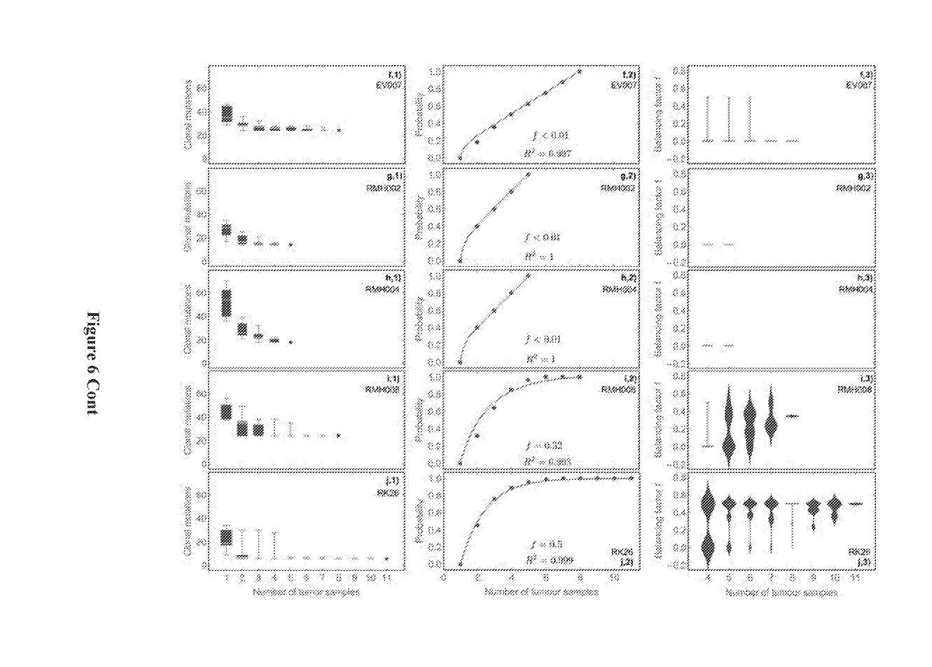

We contemplate that to sample from both sides of the tree, all alterations that appear clonal in both samples will also be truly clonal in the whole tumors. But if we only sample from either side, we will misclassify a fraction of sub-clonal alterations as clonal, see FIG. 5. Thus, how likely are we to sample from both sides of the tree in a multi-sampling strategy?Assuming we analyzed i independent spatially separated tumor samples, the probability to sample from both sides of the tree is

p_{f}(i)=1−f¹−(1−f)³, (8).

The information gained from multi-region sequencing follows a single universal curve (7) and the balancing factor f determines the shape of this curve, see FIG. 5d. The probability to classify all truly clonal alterations correctly from a single sample is expected to be zero (pf(i=1)=0). Including more samples i to the analysis increases the probability to classify truly clonal alterations correctly. The probability increases fastest for trees in which the first bifurcation splits the tumor population approximately in half (f=½). These are often referred to as ‘balanced’ phylogenetic trees, and are often, but not always, consistent with neutral growth (i.e. all the tumor driving alterations were present in the trunk of the tree) (27). In this case, the information is gained exponentially with the number of samples i. Two tumor samples have a probability of 50% to correctly classify all truly clonal alterations and the probability increases to 99% for 8 independent samples. However, the probability increases more slowly in unbalanced tumors, e.g. in cases of strong on-going sub-clonal selection during tumor growth or as a result of treatment. For example, if one side of the tree is 5 times larger compared to the other side, two independent tumor samples result in a probability of 28% to correctly classify all alterations and increases to 73% for 8 independent samples (FIG. 5). Given that the spatial distribution of mutations in the tumor cannot be known a priori, there cannot be a unique sampling strategy, as different tumors might present with different relative f and the uncertainty to identify truly clonal alterations might be dramatically different for two patients with the same number of samples. Ideally, the sampling strategy should be adjusted to account for each tumor's individual evolutionary trajectory.

### Example 2

Estimating the Expected Gain of Additional Tumor Samples

Here we propose a simple method to calculate the probability p_{f}(n) to find all clonal mutations from n independent tumor samples. This method allows estimating the balancing factor f, to calculate the expected gain of information by including additional tumor samples in the analysis:
(i) Collect n samples of a tumor.
(ii) Sequence the n samples and determine all mutations that appear clonal in each sample independently.
(iii) Take the intersection of all mutations identified as clonal in all n tumor samples.
(iv) Take the intersection of all mutations identified as clonal for all possible combinations of 1 to n−1 tumor samples.
(v) Calculate the probability that the mutations identified in step (iii) and (iv) coincide.
   By definition, this probability is 0 for n=I and approaches 1 for the combination of all n samples.

To allow a comparison with Equation (1), we have to normalize accordingly and get

$${p}_{f} ( i , n ) = \frac{1 - {f}^{i} - {( 1 - f )}^{i}}{1 - {f}^{n} - {( 1 - f )}^{n}} \qquad ( 7 )$$

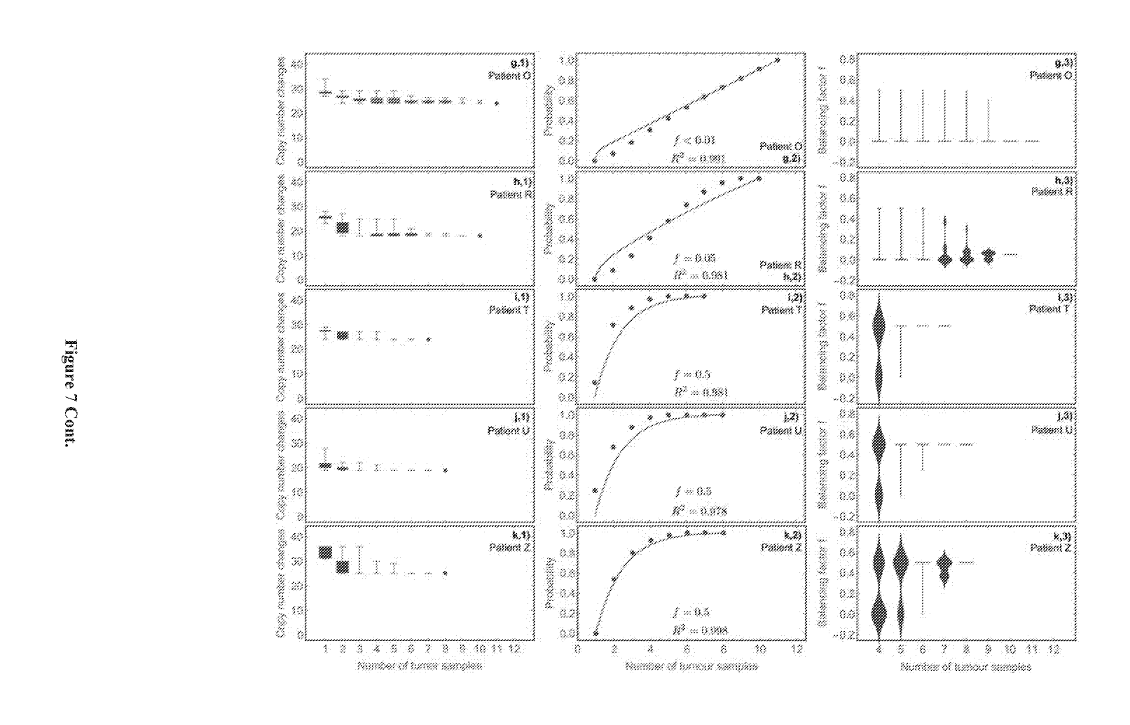

Here n is the maximal number of available samples and i=1, . . . , n denotes possible sub-samples. The only free parameter of this equation is f. Thus a comparison of Equation (7) to actual tumor data allows us to infer f, see for additional examples FIGS. 6 and 7. We use standard least square regression to infer the single free parameter f.

Our algorithm is sensitive to misclassified mutations, e.g. mutations not found in a subset of samples due to normal contamination or limitations of sequencing depth (false negatives). Those are inevitable problems in multi-region sequencing studies, leading to a few mutations that seem to contradict the phylogenetic history of these tumors, the so-called “homoplasy” events. Standard phylogenetic reconstruction algorithms, such as Maximum Parsimony, discard those, hence we filtered the few homoplasy events present in a small subset of renal patients (3/10) from our analysis.

In short, comparing the lists of clonal alterations identified by all permutations of tumor samples gives a measure for the average information gained by additional sampling. This information gain should fall onto the universal curve (8) after adjusting for finite sampling (see Equation (7)). For example, if we have 10 tumor samples in total, we can generate 45 unique combinations of 2 sub-samples. If the tumor were perfectly balanced (f=0.5), half of the sub-sample combinations would recover the exact minimal list of clonal alterations. For unbalanced tumors (f<0.5) fewer combinations of sub-samples will recover the minimal list of alterations. This procedure is then continued for all possible combinations of sub-samples. Comparing the shape of the universal curve (7) to the actual information gain from the data allows assigning an empirical balancing factor f to a tumor. Each tumor-specific balancing factor provides a rational of whether the current number of tumor samples is sufficient, or if additional sampling is necessary to ascertain the identity of truly clonal alterations in that particular patient. In addition, the value of f would determine whether it makes sense to sequence additional parts of the tumor, if the expected information gain from each sample is very small.

To illustrate this observation, we re-analyzed a recently published multi-region sequencing dataset of clear cell renal carcinoma (17), see Example 3 below.

### Example 3

Multi-Region Sequenced Clear Cell Renal Carcinoma

Recently Gerlinger et al published an analysis of the evolutionary history of ten multi-region sequenced clear cell renal carcinomas (14, 17). This dataset provides a unique opportunity to apply our method. We tested if the information on clonal alterations gained from multi-region sequencing data falls onto the theoretically predicted universal curve (7). The number of independent samples ranged from 5 to 11 samples per tumor, 74 samples in total. Each sample had a volume of approximately 0.25 mm³ and thus each sample contained ˜10⁸ cells. The coding region of the genome was sequenced with a depth of >70× for all samples, allowing the identification of clonal mutations within single bulk samples with high precision. Furthermore, three tumors were treatment naive, six received treatment with the mTOR inhibitor everolimus for six weeks and one tumor was treated with the anti-angiogenic drug sunitinib for 16 weeks prior to nephrectomy (see (17) for details).

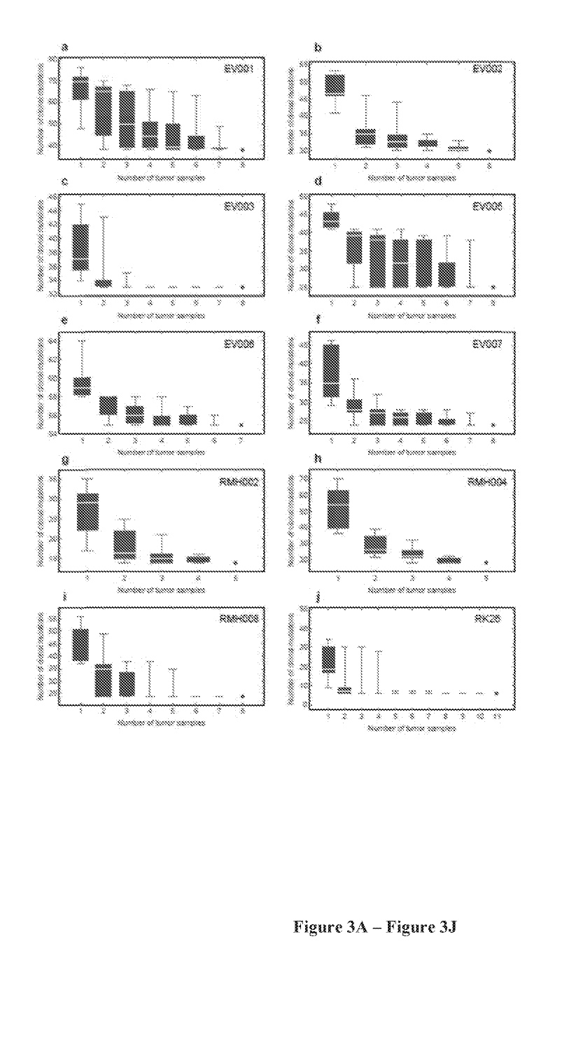

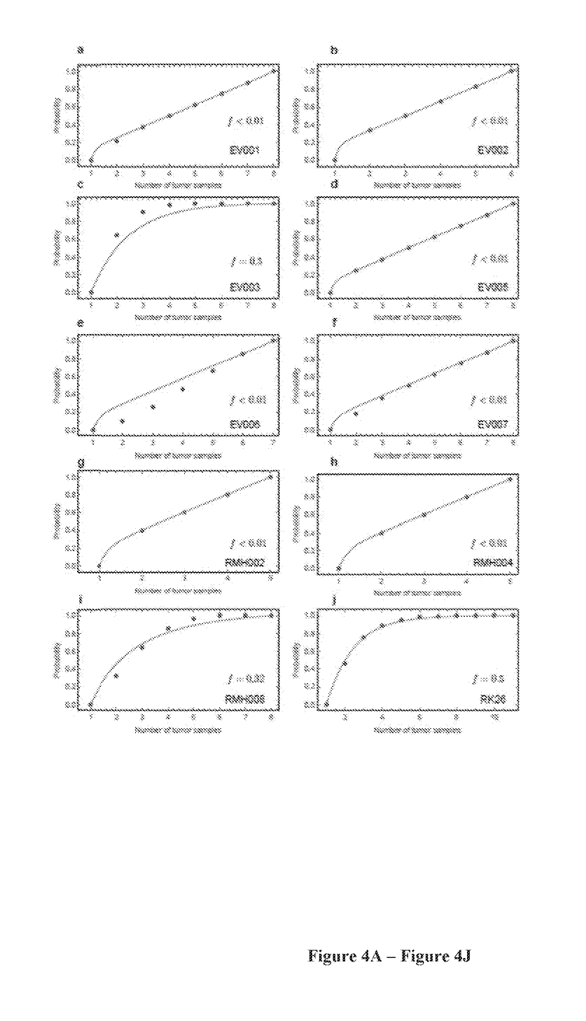

In general, intra-tumor heterogeneity was high in all 10 patients (i.e. 10 tumors). After correcting for homoplasy events, the number of clonal coding mutations identified within a single sample ranged from 9 to 76 across tumors, see FIG. 6 panel a1 to j1, and the variance was high within all tumors, see FIG. 3. Considering more samples in the analysis decreases the number of what appeared to be clonal mutations, as well as the variability in all cases, e.g. 8 samples from the same tumor reduced the list of clonal mutations on average by 40% compared to a single sample and the reduction ranged from 14 to 72% in individual patients, see also FIG. 6 panel a1 to j1.

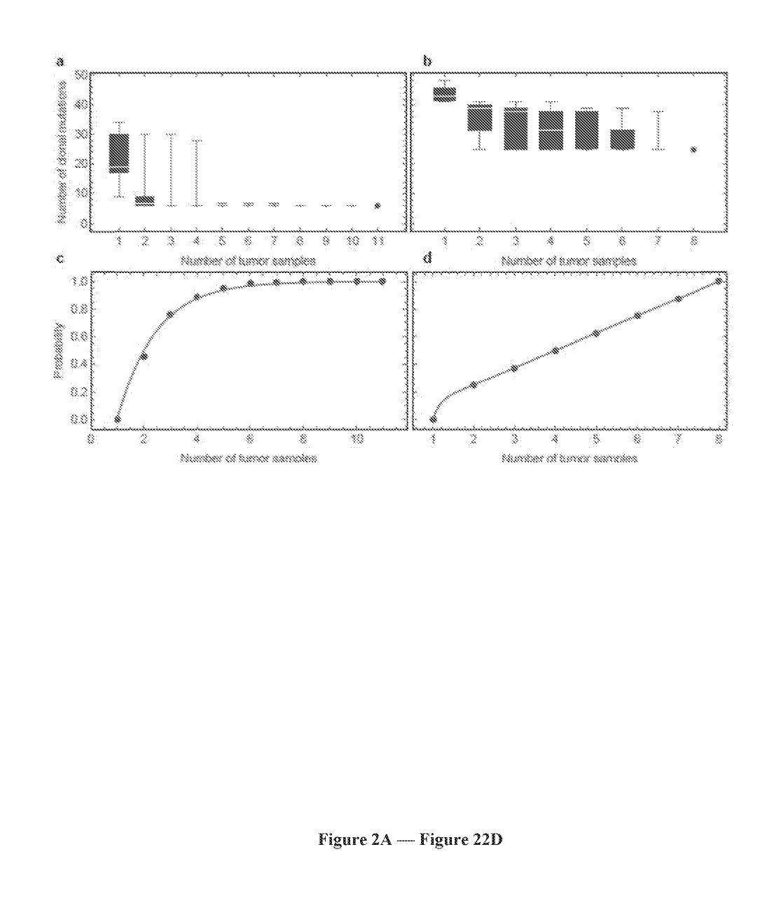

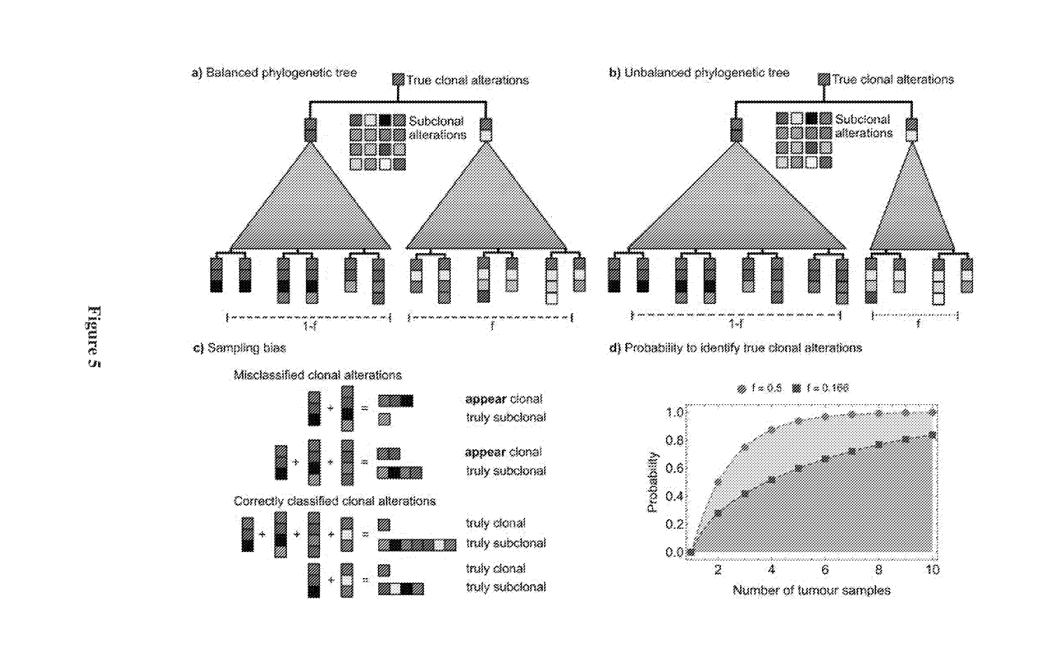

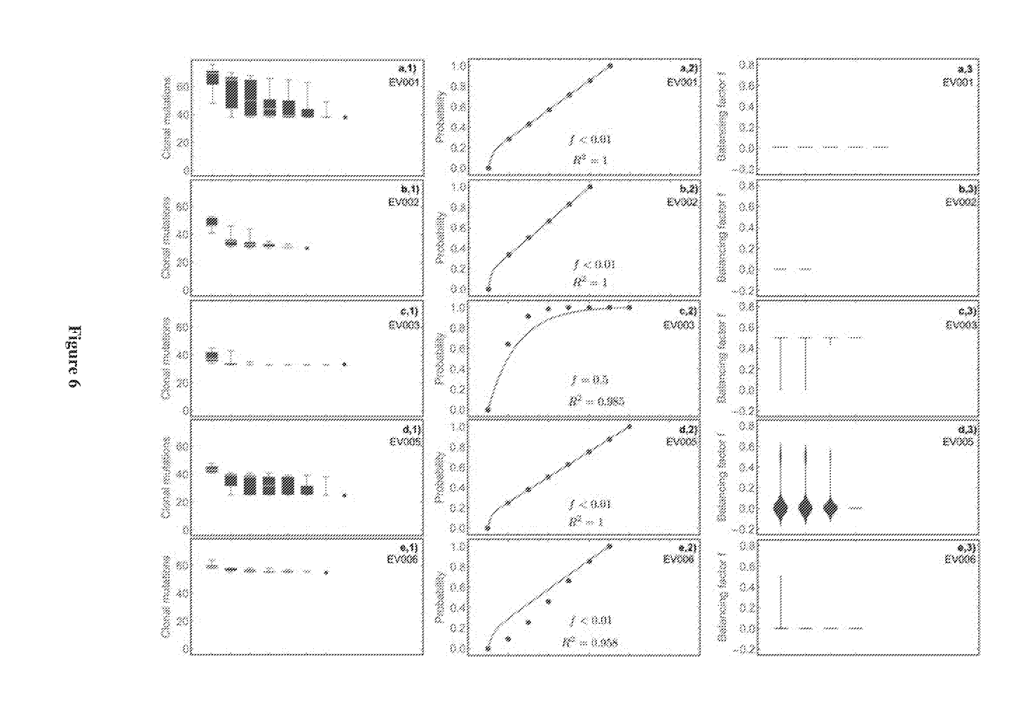

As expected, adding additional samples to the analysis decreases the number of clonal mutations as well as the variance in all 10 cases. The minimum number of clonal exonic mutations across all samples of a tumor ranged from as few as 6 in one patient to up to 55 in another patient, but typically was between 25 to 35. We next applied our algorithm (i)-(v) in each patient (see FIG. 4) and calculated the probability to find the minimal set of clonal mutations. A single sample never identifies the minimal set of clonal mutations and the probability is 0 accordingly. The probability increases steadily with additional tumor samples, but the patterns differ between patients, see FIG. 2 for two examples.

By fitting Equation (7) to the inferred probabilities we estimate f for each patient. We find a balanced tree (f˜½) in 2 cases, see FIG. 4. In other words, none of the subclonal coding mutations introduced a significant fitness differential in these cases. Here 8 independent tumor samples give a probability of 99% to identify all true clonal mutations (see Equation (4)). Also, the variance between samples declines fast and is in accordance with an exponentially decreasing information gain with additional samples (Equation (3). One case is between a highly balanced and unbalanced left and right side (f=0.32), while 7 cases showed a highly unbalanced left and right side with f=0.01, . . . , 0.1. In the latter cases, tumor expansion is likely driven by on-going clonal selection, confirming the original findings of the authors of on-going clonal selection and convergent evolution in the majority of the patients analysed (14, 17). This is also shown by the remaining high variance of clonal mutations between different combinations of sub-samples. Thus a study with fewer samples on the same tumors would identify very different sets of clonal mutations. It is important to point out that the minimal number of identified clonal mutations is not a reliable estimate for the potential gain of further information by additional samples, see for example FIG. 3. Several highly unbalanced cases identify a minimum set around 20 clonal mutations (FIG. 3 d,f,g,h), as do two neutral cases (FIG. 3 c,i). Also a simple comparison to known oncogenes might be misleading, as they also can be repeatedly found in healthy tissues and thus might not always be responsible for the tumor phenotype (13, 22, 23).

Interestingly, 6 out of 7 highly unbalanced tumors were resected after treatment and all 7 cases developed metastatic disease. In contrast 2 out of 3 (nearly) neutral tumors were treatment naive and the only 2 tumors without metastatic disease (RMH008 and RK26) were identified neutral by our approach. Treatment introduces a high selection pressure and it is thus not surprising that treatment leads to highly unbalanced tumors. It poses an interesting question, if neutral tumors are less aggressive, explaining the two cases of metastasis free patients, or if neutrality is a general future of untreated tumors.

### Example 4

The Universal Curve (7) Describes the Information Gain from Additional Samples.

Surprisingly, the universal curve (7) describes the information gain from additional samples very well in all 10 cases and we can assign balancing factors to all 10 tumors. We found balanced phylogenetic trees (f=0.5) in only two tumors; see FIG. 6 panel a2 to j2. In these cases, eight tumor samples suffice to identify all truly clonal mutations with a probability of 99%. One tumor had a slightly unbalanced tree (f=0.35), while 7 tumors appeared to be highly unbalanced (f<0.01). In the latter cases, distinct clonal expansions were likely driven by selection, supporting the original findings of the authors of on-going clonal selection and convergent evolution in the majority of the patients analyzed (Geriinger M, et al. (2012) Intratumor Heterogeneity and Branched Evolution Revealed by Multiregion Sequencing. New England Journal of Medicine 366(10):883-892; Gerlinger M, et al. (2014) Genomic architecture and evolution of clear cell renal cell carcinomas defined by multiregion sequencing. Nature Genetics 46(3):225-233).

In these cases, a study with fewer or different samples on the same tumor would have identified very different sets of clonal mutations. Based on the data, two samples have a median probability of 68% (a 95% CI of 55 to 77%) to overestimate the number of clonal mutations, highlighting the potential risk of suboptimal treatment strategies due to incomplete information on clonal genomic changes of tumor cells. Adding more tumor samples to the analysis of the 7 unbalanced tumors would likely reduce the list of putative clonal mutations further, allowing for a better-informed course of treatment.

We note that the balancing factor f was independent from the total number of uniquely detected mutations (Spearman Rho=−0.38, p=0.3), or the percentage of uniquely detected mutations defined as clonal across all samples of a single tumor (Spearman Rho=0.18, p=0.62). The mutational load of a tumor is the result of many potentially interacting factors, e.g. the age of a patient or the intrinsic (potentially elevated) mutation rate. Furthermore the majority of mutations are likely neutral passengers or provide only a weak selective advantage to the tumor and correlations might be masked by treatment induced selection biases. This suggests that a sampling strategy based on mutational diversity alone may not be optimal. As we show here, the change of diversity across independent tumor samples is one variable of interest.

### Example 5

Analysis of a Subset of Tumor Samples.

We then tested the robustness of our estimates by applying our analysis to a subset of tumor samples. We inferred the balancing factor f for all possible combinations of subsets with a minimum of 4 samples. For example, all combinations of 6 out of 12 tumor samples yield 924 independent estimates for f. The distributions of values for f are summarized in FIG. 6 panel a3 to j3. Most combinations of samples resemble the balancing inferred from the full data set. We observe a trend towards a bimodal distribution for small sample numbers (e.g. FIG. 6 d3, i3 and j3). This might be a direct consequence of the spatial sampling scheme. Few samples in close spatial proximity are more likely to show balanced (neutral) growth characteristics, whereas samples with maximal spatial distance likely diverged early during tumor development (Sottoriva A, et al. (2015) A Big Bang model of human colorectal tumor growth. Nature Genetics. doi:10.1038/ng.3214; 27; 28). This suggests that conclusions about the evolutionary history of tumors based on only a few samples can be misleading. Sufficiently many spatially distant tumor samples are required for a reliable inference (and interpreted in the context of f).

Surprisingly, 6/7 unbalanced tumors received treatment before resection (and sequencing) and all 7 cases developed metastatic disease. In contrast, 2/3 balanced tumors were treatment naive at the time of sequencing and the only 2 tumors without metastatic disease (FIG. 6i,j) were balanced. Indeed, tree unbalancing was associated with treatment (p=0.02, t-test), indicating that treatment likely contributes to high selection pressures that lead to unbalanced phylogenetic structures. This has important biological and clinical implications, suggesting that treated tumors may require more samples to design the optimal therapeutic strategy based on truly clonal alterations. In addition, it appears that multi-region sequencing before initiation of any therapy may simplify the identification of truly clonal abnormalities that could be the targets of therapy. Future studies are needed to test this observation further. It will also be important to stratify patients for potentially other confounding factors, such as tumor size, tumor stage, and the spatial distribution of tumor samples.

### Example 6

Copy Number Changes in Patients with Clear Cell Renal Carcinoma and Colorectal Cancer Follows Our Theoretical Prediction.

We also wanted to find out whether the information on copy number changes in tumors also follows our theoretical prediction (7). We reevaluated copy number changes in multiple single crypts (each crypt contains ˜10⁴ cells) of 11 treatment naive colorectal tumors (7-13 crypts per tumor, 10⁷ samples in total) previously published in (Sottoriva A, et al. (2015) A Big Bang model of human colorectal tumor growth. Nature Genetics. doi:10.1038/ng.3214). Again the information gain from multiple tumor samples is well described by our theoretical model (see FIG. 7 panels a2 to k2). Five tumors are characterized by balanced phylogenetic trees (f≈0.5), two cases show slightly unbalanced trees (f=0.19 and f=0.3) and four cases have unbalanced trees (f<0.01). Based on this data, two samples have a median probability of 58% (95% CI of 38 to 75%) to overestimate the number of clonal copy number changes. Overall, these results support previous observations of largely a single clonal expansion in a majority of colorectal tumors that would lead to more balanced phylogenetic trees (Sottoriva A, et al. (2013) Intratumor heterogeneity in human glioblastoma reflects cancer evolutionary dynamics. Proceedings of the National Academy of Science 110:4009-4014; 27). In these cases, a few samples can identify truly clonal copy number changes. However, we also identified four cases with an unbalanced phylogenetic history, similar to the 7 cases in renal cell carcinoma. Treatment strategies for these patients might benefit from an analysis of additional samples.

There was no observed correlation between tumor balancing and the total number of unique copy number changes (Spearman Rho=0.16, p=0.63). However, we observed a strong positive correlation between the balancing factor f and the percentage of unique copy number changes (Spearman Rho=0.76, p=0.007). Balanced tumors (f=0.5) acquired fewer sub-clonal copy number changes (relative to the number of clonal copy number changes) compared to unbalanced tumors. This is in contrast to the mutational burden in renal cancer patients, where we could not observe a similar correlation. There are several potential reasons for this observation. Colon cancer samples were treatment naive. Copy number changes occur less frequently compared to mutations and do not accumulated with age in healthy tissues. Furthermore it seems plausible that a larger fraction of copy number changes is under selection (either positive or negative), whereas the majority of mutations are likely neutral passengers. The balancing estimates on all possible combinations of tumor samples yield results similar to the mutational burden in renal cancer (FIG. 6 panels a2 to j2 and FIG. 7 panels a2 to k2). The majority of subsamples resemble balancing estimates from the full data set. Again, we observe the trend of a bimodal distribution of the balancing factor f for small numbers of tumor samples.

We note that our analysis does not depend on the detailed effects of selection, i.e. whether selection acts on copy number changes, mutations or epigenetic alterations. Changes in tree balance caused by any type of fitness advantage could potentially be detected. Moreover, the evolutionary mechanisms that generate balanced or unbalanced trees can be arbitrarily complex (29). Our method is agnostic to the specific evolutionary dynamics of the tumor, but instead it leverages on the existing data and in particular on the topology of the phylogenetic tree. Our approach is based on the assumption that multi-region profiling represents the tumor's evolutionary history, e.g. the samples are equally spatially distributed throughout the whole tumor and are not restricted to a small region only.

### REFERENCES

1. Vogelstein B, et al. (2013) Cancer Genome Landscapes. Science 339:1546-1558.
2. Chin L, Andersen J N, Futreal P A (2011) Cancer genomics: from discovery science to personalized medicine. Nat Med 17(3):297-303.
3. van 't Veer L J, Bernards R (2008) Enabling personalized cancer medicine through analysis of gene-expression patterns. Nature 452(7187):564-570.
4. Sawyers C L (2004) Targeted cancer therapy. Nature 432:294-297.
5. Schrama D, Reisfeld R A, Becker J C (2006) Antibody targeted drugs as cancer therapeutics. Nat Rev Drug Discov 5(2):147-159.
6. Bozic I, et al. (2013) Evolutionary dynamics of cancer in response to targeted combination therapy. eLife 2:e00747.
7. Gatenby R A, Silva A S, Gillies R J, Frieden B R (2009) Adaptive Therapy. Cancer Research 69(11):4894-4903.
8. Yates L R, Campbell P J (2012) Evolution of the cancer genome. Nature Reviews Genetics 13(11):795-806.
9. Burrell R A, McGranahan N, Bartek J, Swanton C (2013) The causes and consequences of genetic heterogeneity in cancer evolution. Nature 501(7467):338-345.
10. Marusyk A, Almendro V, Polyak K (2012) Intra-tumor heterogeneity: a looking glass for cancer? Nature Publishing Group 12(5):323-334.
11. Greaves M, Maley C C (2012) Clonal evolution in cancer. Nature 481(7381):306-313.
12. Swanton C (2012) Intratumor Heterogeneity: Evolution through Space and Time. Cancer Research 72(19):4875-4882.
13. Morrissy A S, et al. (2016) Divergent clonal selection dominates medulloblastoma at recurrence. Nature 529:351-357.
14. Gerlinger M, et al. (2012) Intratumor Heterogeneity and Branched Evolution Revealed by Multiregion Sequencing. New England Journal of Medicine 366(10):883-892.
15. Sottoriva A, et al. (2013) Intratumor heterogeneity in human glioblastoma reflects cancer evolutionary dynamics. Proceedings of the National Academy of Science 110:4009-4014.
16. Martincorena I, et al. (2015) High burden and pervasive positive selection of somatic mutations in normal human skin. Science 348:880-886.
17. Gerlinger M, et al. (2014) Genomic architecture and evolution of clear cell renal cell carcinomas defined by multiregion sequencing. Nature Genetics 46(3):225-233.
18. Sottoriva A, et al. (2015) A Big Bang model of human colorectal tumor growth. Nature Genetics. doi: 10.1038/ng.3214.
19. de Bruin E C, et al. (2014) Spatial and temporal diversity in genomic instability processes defines lung cancer evolution. Science 346:251-256.
20. Ling S, et al. (2015) Extremely high genetic diversity in a single tumor points to prevalence of non-Darwinian cell evolution. Proceedings of the National Academy of Sciences. doi:10.1073/pnas.1519556112.
21. Siegmund, K. & Shibata, D. At least two well-spaced samples are needed to genotype a solid tumor. BMC Cancer 26, 1-8 (2016).
22. Genovese G, et al. (2014) Clonal Hematopoiesis and Blood-Cancer Risk Inferred from Blood DNA Sequence. New England Journal of Medicine 371 (26):2477-2487.
23. Yoshizato T, et al. (2015) Somatic Mutations and Clonal Hematopoiesis in Aplastic Anemia. New England Journal of Medicine 373(1):35-47.
24. Chapman P B, et al. N Engl J Med. 2011; 364:2507
25. Kwak E L, et al. N Engl J Med. 2010; 363:1693
26. Altrock, P. M., Liu, L. L. & Michor, F. The mathematics of cancer: integrating quantitative models. Nature Reviews Cancer 15, 730-745 (2015).
27. Williams M J, Werner B, Barnes C P, Graham T A, Sottoriva A (2016) Identification of neutral tumor evolution across cancer types. Nature Genetics 48:238-244.
28. Waclaw, B. et al. A spatial model predicts that dispersal and cell turnover limit intratumour heterogeneity. Nature 525, 261-264 (2015).
29. Heard, S. B. Patterns in Tree Balance among Cladistic, Phenetic, and Randomly Generated Phylogenetic Trees. Evolution 46, 1818-1826 (1992).
30. Welch, J. S. et al. TP53 and Decitabine in Acute Myeloid Leukemia and Myelodysplastic Syndromes. New England Journal of Medicine 375, 2023-2036 (2016).
31. Ford, A. M. et al. Origins of ‘late’ relapse in childhood acute lymphoblastic leukemia with TEL-AML1 fusion genes. Blood 98, 558-558 (2001).
32. Ford, A. M. et al. Protracted dormancy of pre-leukemic stem cells. Leukemia 29, 2202-2207 (2015).

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for identifying a true clonal mutation in cancer cells from a mammal for use in treating said mammal, comprising,
A) generating whole exome sequencing data from at least three samples of cancer cells obtained from different regions in said mammal, comparing said sequencing data to corresponding wild-type reference genes in a databank, and thereafter:
i) Identifying the mutated genes in common between all three samples and identifying mutated genes that are in common between any two sample combinations, and
ii) determining whether said mutated genes in common between all three samples are the same as the mutated genes in common between said two sample combinations, wherein when the number of mutated genes in common with all three samples is equal to or greater than the number of mutated genes in common between said two sample combinations then mutated genes in common in all three samples are true clonally mutated genes, then proceed to step B), and
B) administering to said mammal a therapeutically effective amount of a compound targeting said cancer cells containing at least one said true clonally mutated genes for treatment of said cancer cells.

2. The method of claim 1, wherein at least one of said identified true clonally mutated genes is a tyrosine kinase gene.

3. The method of claim 2, wherein said compound is a tyrosine kinase inhibitor.

4. The method of claim 1, wherein at least one of said true clonally identified genes is an epidermal growth factor receptor (EGFR) gene and wherein at least one of said true clonally identified mutated genes is not a KRAS gene.

5. The method of claim 4, wherein said compound is an anti-EGFR1 therapeutic monoclonal antibody.

6. The method of claim 1, wherein said treatment slows cancer cell growth in said patient.

7. The method of claim 1, wherein said treatment kills cancer cells in said patient.

8. The method of claim 1, wherein when the number of mutated genes in common with all three samples is less than the number of mutated genes in common between said two sample combinations then generating whole exome sequencing data from an additional sample of cancer cells obtained from different regions in said mammal, comparing said sequencing data to corresponding wild-type reference genes in a databank, and thereafter:
iii) Identifying the mutated genes in common between all four samples and identifying mutated genes that are in common between any three sample combinations,
iv) determining whether said mutated genes in common between all four samples are the same as the mutated genes in common between said three sample combinations, wherein when the number of mutated genes in common with all four samples is equal to or greater than the number of mutated genes in common between said three sample combinations then mutated genes in common in all four samples are true clonally mutated genes, then proceed to step B).

9. A method for treating cancer in a mammalian subject in need thereof, comprising,
A) identifying a mutation in said cancer as a clonal mutation, wherein said identifying comprises,
i) collecting n independent samples from said cancer, wherein n is an integer number greater than 1,
ii) sequencing the n samples and determining all clonal mutations in each sample independently,
iii) taking the intersection of all mutations identified as clonal in all n cancer samples,
iv) taking the intersection of all mutations identified as clonal for all combinations of 1 to n−1 cancer samples, and
v) calculating the probability that the mutations identified in steps (iii) and (iv) coincide, wherein a calculated probability of at least 99% for a mutation identifies said mutation as a clonal mutation, and
B) administering to said subject a therapeutically effective amount of a compound that alters expression of said clonal mutation, thereby treating said cancer.
